(19) United States
(12) Patent Application Publication
(10) Pub. No.: US 2008/0280994 A1
(43) Pub. Date: Nov. 13, 2008

(54) Ascophyllum Compositions and Methods

(75) Inventors: Junzeng Zhang, Halifax (CA); Harry Stephen Ewart, Halifax (CA); Jing Kai Shen, Halifax (CA); Colin James Barrow, Halifax (CA)

Correspondence Address: SMART & BIGGAR;P.O. BOX 2999, STATION D, 900-55 METCALFE STREET, OTTAWA, ON, K1P5Y6, CA

(73) Assignee: OCEAN NUTRITION CANDA LTD.

(21) Appl. No.: 11/660,275

(22) PCT Filed: Aug. 15, 2005
(86) PCT No.: PCT/CA2005/001323

§ 371 Date: Feb. 16, 2007

Related U.S. Application Data
(60) Provisional application No. 60/601,971, filed on Aug. 17, 2004.

(51) Int. Cl. A61K 31/085; C07C 39/12; C07C 43/20; A61P 3/10; C08B 37/00; A61K 31/05

(52) U.S. Cl. 514/721; 568/730; 568/638; 536/123.1; 514/736

(57) ABSTRACT

The invention provides extracts of brown seaweed of the genus Ascophyllum that have valuable pharmacological properties. In particular, compositions of the invention are useful for e.g.: inhibiting alpha-glucosidase activity; preventing or treating conditions mediated by alpha-glucosidase activity; reducing blood glucose levels; preventing or treating diabetes; modulating glucose uptake in adipocytes; preventing or treating obesity; scavenging free radicals; stimulating the immune system; activating macrophages; preventing or treating condition mediated by macrophage activation; and modulating nitric oxide production by macrophages. Methods for using the Ascophyllum extracts of the invention are provided, as are kits comprising Ascophyllum extracts of the invention and instructions for using the extracts.

### CROSS REFERENCE TO RELATED APPLICATIONS

This application claims the benefit of U.S. Provisional Patent Application No. 60/601,971, filed Aug. 17, 2004, which is incorporated herein by reference.

### FIELD OF THE INVENTION

The invention concerns extracts of Ascophyllum, particularly Ascophyllum nodosum, and compositions comprising such extracts, and their medicinal use.

### BACKGROUND OF THE INVENTION

There are two major types of diabetes mellitus: type I and type II diabetes. Type I diabetes mellitus is also called insulin dependent diabetes mellitus (IDDM), or juvenile onset diabetes mellitus.

In type I diabetes mellitus, the pancreas undergoes an autoimmune attack by the body itself, and is rendered incapable of making insulin. Abnormal antibodies have been found in patients with type I diabetes. Antibodies are proteins in the blood that are part of the body's immune system. The patient with type I diabetes must rely on insulin administration by, for example, injection for survival.

In type II diabetes [also referred to as non-insulin dependent diabetes mellitus (NIDDM) or adult onset diabetes mellitus (AODM)], patients can still produce insulin, but do so relatively inadequately. In many cases this actually means the pancreas produces larger than normal quantities of insulin. A major feature of type II diabetes is a lack of sensitivity to insulin by the cells of the body (particularly fat and muscle cells). In addition to the increase in insulin resistance, the release of insulin by the pancreas may also be defective, and occur late in response to increased glucose levels. Finally, the liver of patients with type II diabetes continues to produce glucose despite elevated glucose levels.

It is known that inhibition of α-glucosidase (an enzyme of the small intestine which catalyses the hydrolysis of terminal, non-reducing 1,4-linked α-D-glucose residues with release of D-glucose) is beneficial to patients with type II diabetes. Pharmaceutical preparations of α-glucosidase inhibitors are available from Bayer under the trade marks Precose™ (acarbose) and Glyset® (miglitol). Acarbose is a complex oligosaccharide of microbial origin and miglitol is a deoxynojirimycin derivative.

The use of pharmaceutical α-glucosidase inhibitors is associated with gastrointestinal side effects, which have limited their use. The most common side effects are temporary digestive symptoms including abdominal discomfort, excessive gas (flatulence), and diarrhoea.

The brown seaweed Ascophyllum nodosum has been used as a significant source of raw material for the alginate industry. Alginates are used as coagulants, in beer production, food production, in filters to remove heavy metals, etc. Dried, pulverised Ascophyllum is used as an animal feed additive and is also used as a raw material for the production of hydrolysates used as plant food or fertiliser.

### BRIEF SUMMARY OF THE INVENTION

In addition to the above uses, which largely take advantage of the physical properties of the seaweed, we have discovered that Ascophyllum extracts have valuable medicinal properties.

According to one aspect of the present invention, there is provided an extract of Ascophyllum, comprising at least about 20% by weight of polyphenolic compounds having an average molecular weight of about 30 kDa to about 830 kDa.

According to another aspect of the present invention, there is provided an extract obtained or obtainable by an extraction process comprising extracting Ascophyllum with an aqueous organic solvent to form an aqueous organic extract.

According to another aspect of the present invention, there is provided a method for preparing an extract of Ascophyllum, comprising extracting Ascophyllum with an aqueous organic solvent to form an aqueous organic extract.

According to another aspect of the present invention, there is provided an extract of Ascophyllum, comprising at least about 40% by weight of sulfated polysaccharides having a molecular weight of about 100 kDa to about 3000 kDa.

According to still another aspect of the present invention, there is provided an extract obtained or obtainable by an extraction process comprising extracting Ascophyllum with water to form an aqueous extract.

According to another aspect of the present invention, there is provided a method for preparing an extract of Ascophyllum, comprising extracting Ascophyllum with water to form an aqueous extract.

According to yet another aspect of the present invention, there is provided a composition comprising an extract as described herein.

According to further aspects of the present invention, there are provided methods for using an extract or composition described herein for inhibiting alpha-glucosidase activity; preventing or treating conditions mediated by alpha-glucosidase activity; reducing blood glucose levels; preventing or treating diabetes; modulating glucose uptake in adipocytes; preventing or treating obesity; scavenging free radicals; stimulating the immune system; preventing or treating a condition mediated by macrophage activation; and modulating nitric oxide production by macrophages.

According to yet a further aspect of the present invention, there is provided a kit comprising an extract or a composition described herein, and instructions for using said extract or composition in the above-described methods.

### BRIEF DESCRIPTION OF THE DRAWINGS

FIG. 1 depicts the results of a maltose challenge in mice with fraction JK02202.

FIG. 2 depicts the results of a glucose-uptake assay for fraction JK02202.

FIG. 3 depicts a proton NMR spectrum of fraction JK02206.

FIG. 4 depicts a carbon NMR spectrum of fraction JK02206.

FIG. 5 depicts an IR spectrum of fraction JK02206.

FIG. 6 depicts an IR spectrum of fraction JZ07942.

FIG. 7 depicts a proton NMR spectrum of fraction JZ07942.

FIG. 8 depicts a carbon NMR spectrum of fraction JK07942.

FIG. 9 depicts an IR spectrum of fraction JZ07943.

FIG. 10 depicts a proton NMR spectrum of fraction JZ07943.

FIG. 11 depicts the results of a glucose-uptake assay for fraction JZ07942.

FIG. 12 depicts a proton NMR spectrum of fraction JK02942.

FIG. 13 depicts a proton NMR spectrum of fraction JK02741.

FIG. 14 depicts a carbon NMR spectrum of fraction JK02741.

FIG. 15 depicts an IR spectrum of extract ON169.3a.

FIG. 16 depicts an IR spectrum of extract ON169.3a.

FIG. 17 depicts the blood glucose lowering effect of ON169.3a in mice.

FIG. 18 depicts the immune stimulating effects of ON169.4a and fractions thereof in a macrophage activation assay.

FIG. 19 depicts an IR spectrum of extract JZ07734.

FIG. 20 depicts a proton NMR spectrum of extract JZ07734.

FIG. 21 depicts a proton NMR spectrum of fraction JZ07892.

FIG. 22 depicts an IR spectrum of fraction JZ07901.

FIG. 23 depicts a proton NMR spectrum of fraction JZ07901.

FIG. 24 depicts an IR spectrum of extract JZ07932.

FIG. 25 depicts a proton NMR spectrum of extract JZ07932.

FIG. 26 depicts an IR spectrum of fraction JZ07971.

FIG. 27 depicts a proton NMR spectrum of fraction JZ07971.

FIG. 28 depicts a carbon NMR spectrum of fraction JZ07971.

FIG. 29 depicts the blood glucose lowering effects of fractions JZ07932, JZ07901 and JZ07971.

FIG. 30 depicts a proton NMR spectrum of an Ascophyllum extract obtained by extraction with 50% aqueous ethanol solution.

FIG. 31 depicts a proton NMR spectrum of an Ascophyllum extract after degradation with sodium metal in liquid ammonium.

FIG. 32 depicts a proton NMR spectrum of an Ascophyllum extract after acetylation.

FIG. 33 depicts an expanded view of the downfield region of the proton NMR spectrum for the first fraction collected in Example 4.

FIG. 34 depicts a proton NMR spectrum for the first fraction collected in Example 4.

FIG. 35 depicts a LC-MS spectrum, m05511a2, for the first fraction collected in Example 4.

FIG. 36 depicts a LC-MS spectrum, m05511a5, comprising a fragment having an exact mass of 169.079.

FIG. 37 depicts positive and negative ion ESI spectra, m05512a6 and m05512a3, comprising fragments having exact masses of 304.136 and 334.153.

FIG. 38 depicts a proton NMR spectrum for the fraction having a mass spectrum comprising a fragment having an exact mass of 304.136.

FIG. 39 depicts a COSY NMR spectrum for the fraction having a mass spectrum comprising a fragment having an exact mass of 304.136.

FIG. 40 depicts positive and negative ion ESI spectra, m05512a4 and m05512a5, comprising fragments having exact masses of 306.110 and 320.126.

FIG. 41 depicts positive and negative ion ESI spectra, m05510a4 and m05510a5, comprising a fragment at 459.2 m/z.

### DETAILED DESCRIPTION OF THE INVENTION

### Ascophyllum Species and Sources

Suitable Ascophyllum species include e.g. Ascophyllum nodosum, Ascophyllum laevigatum and Ascophyllum mackayi.

Preferred is Ascophyllum nodosum, commonly known as “knotted wrack or rockweed”. A. nodosum is a common large brown seaweed, that is dominant on sheltered rocky shores. The species has long strap-like fronds 0.5 to 2 m in length, with large egg-shaped air bladders at regular intervals. The species attaches to rocks and boulders on the middle shore in a range of habitats, from estuaries to relatively exposed coasts. Although subtidal populations have been reported, an intertidal habit is more usual.

Although not farmed, Ascophyllum nodosum is harvested on a commercial scale in many countries, including Canada, China, Ireland, Iceland, Norway, the UK, the United States, and France. More specifically, Ascophyllum nodosum can be readily collected from the Nova Scotia coast of Canada. Commercial sources include, e.g. Acadian Seaplants Ltd. (Dartmouth, Nova Scotia, Canada) and Maine Coast Sea Vegetables, Inc. (Franklin, Me., U.S.).

### Extracts

The extracts of the invention may comprise, for example, at least about 20% to about 100% by weight of polyphenolic compounds. In particular the extracts may comprise at least 20+1n % by weight polyphenolic compounds, where n is an integer from 0 to 80. In some embodiments, the extracts comprise at least about 20, 30, 40, 50, 60, 70, 80, 90 or 95% by weight of polyphenolic compounds. Weight percent of polyphenolic compounds is calculated as the total mass of polyphenolic compounds divided by the total mass of the extract, wherein the term “extract” refers only to Ascophyllum-derived materials. For instance, in the case of a composition comprising a carrier, diluent, or excipient or the like, together with an Ascophyllum extract, polyphenolic content is determined without regard to the mass of the carrier, diluent, or excipient.

The term “polyphenolic compound” refers to a compound having a backbone comprising phenol as a repeating unit linked by an ether bond or a carbon-carbon bond. The carbon-carbon bond may be in the ortho, meta, or para position relative to the hydroxyl group of the phenol repeating unit. The phenol may be unsubstituted or substituted in the ortho, meta, and/or para positions. The phenol may be substituted by, for example, an unsubstituted or substituted alkyl, an unsubstituted or substituted cycloalkyl, an unsubstituted or substituted alkenyl, an unsubstituted or substituted alkynyl, an unsubstituted or substituted aryl, an unsubstituted or substituted arylalkyl, an unsubstituted or substituted heteroaryl, or an unsubstituted or substituted acyl.

The term “alkyl” or “unsubstituted alkyl”, refers to a branched or unbranched, saturated aliphatic hydrocarbon group having a number of specified carbon atoms, or if no number is specified, having up to and including 12 carbon atoms. For example, methyl, ethyl, n-propyl, isopropyl, n-butyl, iso-butyl, sec-butyl, tert-butyl, n-pentyl, 2-methylbutyl, 2,2-dimethylpropyl, n-hexyl, 2-methylpentyl, 2,2-dimethylbutyl, n-heptyl, 3-heptyl, 2-methylhexyl, and the like.

The term “substituted alkyl” refers to the above alkyl groups substituted by the substituents including, but not limited to, a halogen, a hydroxy, a C₁-C₇ alkoxy, a protected hydroxy, an amino(including alkyl and dialkyl amino), a protected amino, a C₁-C₇ acyloxy, a C₃-C₇ heterocyclyl, a phenoxy, a nitro, a carboxy, a protected carboxy, a carboalkoxy, an acyl, a carbamoyl, a carbamoyloxy, a cyano, a methylsulfonylamino, a benzyloxy, or a C₃-C₆ carbocyclyl. The substituted alkyl may be substituted once, twice or three times per carbon atom with the same or with different substituents.

The term “unsubstituted or substituted cycloalkyl” refers to a mono-, bi-, or tricyclic aliphatic ring having 3 to 14 carbon atoms and preferably 3 to 7 carbon atoms. The cycloalkyl may be optionally substituted by the same substituents as the “substituted alkyl”.

The term “unsubstituted or substituted alkenyl” refers to a branched or unbranched hydrocarbon group having a number of specified carbon atoms, containing one or more carbon-carbon double bonds, each double bond being independently cis, trans, or a nongeometric isomer. The alkenyl group may be optionally substituted by the same substituents as the “substituted alkyl”.

The term “unsubstituted or substituted alkynyl” refers to a branched or unbranched hydrocarbon group having a number of specified carbon atoms, containing one or more carbon-carbon triple bonds. The alkynyl may be optionally substituted by the same substituents as the “substituted alkyl”.

The term “unsubstituted or substituted aryl” refers to a homocyclic aromatic group whether or not fused having a number of specified carbon atoms or if no number is specified, from 6 to 14 carbon atoms. The aryl group may be optionally substituted by the same substituents as the “substituted alkyl” or an aryl, a phenyl or heteroaryl group fused thereto.

The term “aralkyl” means one, two, or three aryl groups having a number of specified carbon atoms, appended to an alkyl group having a number of specified carbon atoms, for example, benzyl. The aralkyl group may be optionally substituted by the same substituents as the “substituted aryl” on the aryl portion and by the same substituents as the “substituted alkyl” on the alkyl portion.

The term “unsubstituted or substituted heteroaryl” refers to any of a mono-, bi-, or tricyclic aromatic ring system having a number of specified atoms where at least one ring is a 5-, 6- or 7-membered ring containing from one to four heteroatoms selected from the group nitrogen, oxygen, and sulfur. The heteroaryl group may be optionally substituted by the same substituents as the “substituted aryl”.

The term “acyl” refers to a group having a number of carbon atoms, appended to a carbon double bonded to an oxygen, for example, formyl, acetyl, propionyl, butyryl, pentanoyl, hexanoyl, heptanoyl, benzoyl and the like.

In a preferred embodiment, the extract may comprise either or both monomers of formulas:

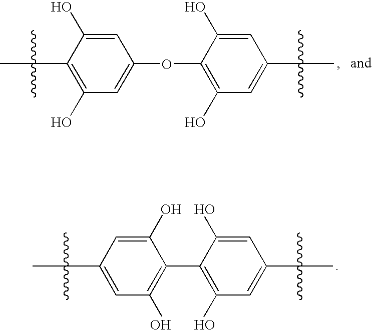

The polyphenolic compounds of the extract described herein may have minimum molecular weights of at least about 40, 50, 60, 70, 80 or 90 kDa, and maximum molecular weights of about 830, 800, 700, 600, 500, 400 or 300 kDa. Ranges bounded by each combination of these minimum and maximum molecular weights are specifically contemplated herein.

In one embodiment, the polyphenolic compounds have a molecular weight in the range of from about 30 kDa to about 830 kDa, more preferably from about 40 kDa to about 300 kDa.

In addition to having the range of molecular weights described above, the average molecular weight of the polyphenolic compounds in the extract may be, for example, about 100 kDa.

The polyphenolic compounds of the extract described herein may comprise, for example, phloroglucinol-based tannins (phlorotannins). The phlorotannins may have a degree of polymerization (“dp”) of, for example, at least about 200, 300, 400, 500, 600, 700, 800, 900 or 1000, and less than or equal to about 8000, 7000, 6000, 5000, 4000, 3000, or 2000. Ranges bounded by each combination of these minimum and maximum degrees of polymerization are specifically contemplated herein. In one embodiment, the phlorotannins have a degree of polymerization in the range of about 200 to about 8000, or from about 200 to about 3000.

The invention also provides Ascophyllum extracts comprising at least about 40+1n % by weight of sulfated polysaccharides, where n is an integer from 0 to 60. In particular, the extracts may comprise at least about 40, 50, 60, 70, 80 or 90% by weight of sulfated polysaccharides. Weight percent of sulfated polysaccharide is calculated as the total mass of sulfated polysaccharides divided by the total mass of the extract, wherein the term “extract” refers only to Ascophyllum-derived materials. For instance, in the case of a composition comprising a carrier, diluent, or excipient or the like, together with an Ascophyllum extract, sulfated polysaccharide content is determined without regard to the mass of the carrier, diluent, or excipient.

The term “sulfated polysaccharide” refers to an oligomer or polymer of a saccharide, either straight-chained, branched, or cyclic, joined together by glycosidic linkages and functionalized by a sulfate group. The sulfation level is determined by the content of sulfate hemi-ester groups attached to sugar units in sulfated polysaccharides, determined by using turbidimetric method. The saccharide units may be identical or different, and may include, without limitation, glucose, fructose, mannose, galactose, ribose, arabinose, xylose, lyxose, erythrose and threose. The saccharide may be further modified at any hydroxyl group to form, for example, an ester, a carbamate, a carbonate, a phosphinate, a phosphonate, a phosphate, a sulfinate, a sulfite, a sulfonate, or R′O—, wherein R′ is linear or branched chain (C₁-C₂₀)alkyl, hydroxy(C₁-C₂₀)alkyl, carboxy(C₁-C₂₀)alkyl, aryl, or aryl(C₁-C₂₀)alkyl.

The sulfated polysaccharide of the extract described herein may have a molecular weight of, for example, at least about 100, 200, 300, 400 or 500 kDa, and less than or equal to about 3000, 2500, 2000, 1500, 1400, 1300, 1200, 1100 or 1000 kDa. Ranges bounded by each combination of these minimum and maximum molecular weights are specifically contemplated herein. In one embodiment, the sulfated polysaccharides have a molecular weight in the range of about 100 kDa to about 3000 kDa, or in the range of about 300 kDa to about 1000 kDa.

### Extraction Methods

The Ascophyllum source material may be in any suitable form for extraction, such as chopped, ground or pulverized form, but is preferably in the form of a powder, for efficient extraction. The Ascophyllum material may be purchased in powdered form or may be made into a powder by any suitable method known in the art. For small samples, a mortar and pestle is appropriate. For large samples, grinding mills or industrial scale pulverizers are more useful. The skilled person can adapt the extent of grinding or pulverizing as required for the particular extraction method employed.

Prior to extraction, lipophilic components may be removed from the Ascophyllum material by extraction with e.g. hexane for a period of time up to e.g. about 24 hours although longer or shorter times may be used.

An enzymatic pre-treatment process, or microwave or sonication-assisted extraction may be used to increase the yield.

Extraction aids and additives, such as buffers, complexing agents, preservatives, and anti-bacterial agents, as are known in the art, may be used.

### Extraction Process for an Extract Enriched in Polyphenolic Compounds

A crude extract of Ascophyllum enriched in polyphenolic compounds may be prepared by extracting Ascophyllum with a solvent, preferably a polar solvent, more preferably a polar solvent miscible with water. Suitable solvents may include, for example, water, ethanol, methanol, 1-propanol, isopropanol, acetone, and aqueous mixtures of these organic solvents in the ratio of, for example, from about 10:90 to about 90:10. Every ratio or range of ratios within this range is specifically contemplated herein, such as, for example, a 50:50 mixture. In a preferred embodiment, an aqueous ethanol solution is employed, more preferably 50% aqueous ethanol solution.

Extraction may be performed at temperatures of, for example, at least about 0, 10, 20, 30, 40, or 50° C. and less than or equal to about 100, 90, 80, 70 or 60° C. Ranges bounded by each combination of these minimum and maximum temperatures are specifically contemplated herein. When extracting with a water/ethanol mixture, such as 50% aqueous ethanol, a temperature in the range of about 25° C. to about 80° C. is preferred, with a temperature of about 50° C. being most preferred. Temperatures in excess of 100° C. may be used, but extractions at these temperatures are typically done under pressure. When extracting with water, a temperature in the range of about 20° C. to about 100° C. is preferred, with a temperature of about 80° C. being most preferred.

The extraction time may be, for example, from about 1 h to about 24 h, from about 2 h to about 6 h, or about 2 h.

The ratio of solvent to Ascophyllum may be, for example, from about 2:1 to about 20:1. Every ratio or range of ratios within this range is specifically contemplated herein. In a preferred embodiment, the ratio of solvent to Ascophyllum is about 4:1 to about 12:1 or about 4:1 to about 12:1.

The extraction may be performed under gentle agitation, which may be achieved by agitating the extraction vessel, by mechanically stirring the slurry or through the action of heating the slurry, and optionally under reflux.

The crude extract may be concentrated through removal of organic solvent, such as by a vacuum method, e.g. rotary evaporation, and then freeze-dried, lyophilized, or spray-dried to yield a powdered crude extract.

The crude extract may be further processed, either before or after concentration and freeze-drying by e.g. filtration, chromatography, dialysis and/or centrifugation, as are known in the art.

Crude extracts may be fractionated through the use of e.g. ultrafiltration, organic solvent partitioning, or the use of chromatography.

The ultrafiltration may be performed with a filter having a membrane with a molecular weight cut-off of, for example, at least about 10, 20, 30, 40, or 50 kDa and less than or equal to about 100, 90, 80, 70 or 60 kDa. Ranges bounded by each combination of these minimum and maximum molecular weight cut-offs are specifically contemplated herein. In one embodiment, the filter has a molecular weight cut-off of about 10, 20, 30, 40, 50, 60, 79, 80, 90, or 100 kDa, or a molecular weight cut-off in the range of about 10 to about 100 kDa, and preferably a molecular weight cut-off of about 30 kDa.

The stationary phase in the chromatographic method may be, for example, CELITE, C-2, C-8, C-18, HP-20, AMBERLITE XAD series resins, reverse-phase modified silica gel adsorbents, polymeric adsorption resins or other similar adsorbents, resins or gels, such as phenyl, cyano, diol, amide, amino, AQ polar end-capped C18.

The mobile phase in the chromatographic method may be an aqueous organic solvent of, for example, from about 50, 60, 70, 80, 90 or 100% ethanol:water. Other organic solvents that may be used as eluants include, but are not limited to methanol, 1-propanol, isopropanol, acetone, and tetrahydrofuran (THF), or their aqueous mixtures. Further, chloroform or a solvent system comprising 10% methanol/90% chloroform may be used as the mobile phase.

In one embodiment, powdered crude extract or an aqueous suspension of the crude extract after rotary evaporation is loaded directly onto a column packed with, for example, Celite, C-18, HP-20, or other similar absorbents or resins. Alternatively, the aqueous suspension of the crude extract is mixed with the stationary phase, for example, CELITE, C-18, HP-20, or other similar absorbents or resins, and then loaded into the column. In each case, the column is washed with water for several column volumes. The bioactive components are eluted using an aqueous organic solvent, for example, from about 50% to about 100% ethanol:water. The resulting aqueous organic solution after elution may be concentrated by vacuum methods such as rotary evaporation and then freeze-dried or spray-dried to obtain the bioactive fraction.

In another embodiment, fractions enriched in polymeric phenolic compounds may be obtained through removal of the organic solvent, for example, ethanol, by vacuum based methods such as rotary evaporation. Subsequently, the aqueous suspension of the crude extract is partitioned between e.g. hexane, ethyl acetate, and n-butanol. Other solvents that may be used include petroleum ether and ethyl ether, or other non-polar organic solvents in place of hexane; methyl acetate, dichloromethane, chloroform, sec- or tert-butanol, or other organic solvents with similar properties in place of ethyl acetate and n-butanol.

A combination of techniques can be used for extraction when appropriate, e.g. first fractionating the crude extract by organic solvent partitioning, followed by further separation of the resulting fractions on a C-18 or HP-20 column, or a C-18 column followed by an HP-20 column.

### Extraction Process for Extract Enriched in Sulfated Polysaccharides

A crude extract of Ascophyllum enriched in sulfated polysaccharides may be prepared by extracting Ascophyllum with a solvent, for example water.

Extraction may be performed at temperatures of, for example, at least about 0, 10, 20, 30, 40, or 50° C. and less than or equal to about 100, 90, 80, 70 or 60° C. Ranges bounded by each combination of these minimum and maximum temperatures are specifically contemplated herein. As discussed above, extractions performed at temperatures over 100° C. are generally performed under pressure.

The extraction time may be, for example, from about 1 h to 5 h, or about 2 h.

The extraction may be performed under gentle agitation, which may be achieved by agitating the extraction vessel, by mechanically stirring the slurry or through the action of heating the slurry, and optionally under reflux.

The crude extract may be concentrated by freeze-drying or spray-drying to yield a powdered crude extract.

The extract enriched in sulfated polysaccharide, may be fractionated by ultrafiltration and/or by precipitation in methanol, ethanol, iso-propanol, acetone, a quaternary ammonium salt such as cetyltrimethylammonium chloride or bromide, or a pyridinium salt such as cetylpyridinium chloride or bromide. Preferably, precipitation is followed by centrifugation to recover the precipitate.

Extraction with aqueous organic solvent and then with water, followed by fractionation may also result in an extract enriched in sulfated polysaccharide.

### Uses

Ascophyllum extracts of the invention have been demonstrated to possess a wide range of valuable medicinal properties.

Fractions containing highly polymeric phenolic compounds were demonstrated in vitro to be potent inhibitors α-glucosidase activity in the assay of Sawada et al. (1993) and to induce blood glucose uptake rate by about 100%. Accordingly, extracts of the invention are useful in the treatment of conditions or disorders that involve or are mediated by α-glucosidase activity or abnormal blood glucose levels, including, for example diabetes, type I and type II diabetes, or diabetes that is secondary to other conditions (e.g. glococorticoid-induced insulin resistance). In addition, α-glucosidase inhibition can be beneficial in obesity and cardiovascular disease.

The same fractions were also demonstrated to possess significant antioxidant activity, making them useful wherever a free radical scavenger would be desirable of in the prevention or treatment of conditions or disorders involving oxidation, particularly abnormal or excessive oxidation. Oxidative stress is involved in a number of disorders including dislipidemia, atherosclerosis, diabetic neuropathy and nephropathy, and neurological diseases such as Alzheimers disease and amyotrophic lateral sclerosis (ALS).

Fractions enriched in sulfated polysaccharides demonstrated both blood glucose-lowering activity, such that they are useful in e.g. the prevention and treatment of diabetes, obesity, cardiovascular disease and other conditions as discussed above, and also immune-stimulating activity, particularly macrophage stimulating activity. Accordingly, Ascophyllum extracts comprising sulfated polysaccharides are also useful for e.g. stimulating the immune system, activating macrophages, and preventing or treating conditions mediated by macrophage activation.

The methods of the invention can be practiced in humans, other mammals, birds, fish, etc.

### Compositions

Ascophyllum or Ascophyllum extracts may be used in a wide variety of forms including, for example, pharmaceutical or neutraceutical products, nutritional supplements, food products (e.g. so-called “functional foods”), food ingredients and beverages. The Ascophyllum extract or composition may be microencapsulated in order to improve palatability or processing characteristics of the food or beverage product. Alternatively, Ascophyllum extracts may be used on their own, e.g. as described in the Examples herein.

The term “nutritional supplement” refers to a product intended to supplement the diet by increasing the total dietary intake that may contain one or more of the following dietary ingredients: a vitamin, a mineral, an herb or other botanical, an amino acid, another dietary substance for use to supplement the diet by increasing the total dietary intake; or a concentrate, metabolite, constituent, extract, or combination of any ingredient described herein. A nutritional supplement is not represented for use as a conventional food or as a sole item of a meal or the diet. A nutritional supplement is intended for ingestion in the form of, for example, tablets, capsules, softgels, gelcaps, sachets, liquids, or powders.

Neutraceutical and pharmaceutical formulations may be for enteral (e.g. oral), rectal, parenteral or other modes of administration. The formulations comprise the composition of the present invention in combination with one or more physiologically acceptable ingredients, such as carriers, excipients and/or diluents. Compositions and formulations for oral administration are particularly preferred.

Formulations may be prepared, for example, in unit dose forms, such as tablets, sachets, capsules, dragees, suppositories or ampoules. They may be prepared in a conventional manner, for example by means of conventional mixing, granulating, confectioning, dissolving or lyophilising processes.

To prepare formulations of the present invention in the form of dosage units for oral administration, the Ascophyllum compositions of the present invention may take the form of, for example, granules, tablets, capsules, liquids or dragees prepared together with physiologically acceptable carriers, excipients and/or diluents.

Typical physiologically acceptable ingredients include:

(a) binding agents such as starch (e.g. pregelatinised maize starch, wheat starch paste, rice starch paste, potato starch paste), polyvinylpyrrolidone, hydroxypropyl methylcellulose, gum tragacanth and/or gelatin;
(b) fillers such as sugars (e.g. lactose, saccharose, mannitol, sorbitol), amylopectin, cellulose preparations (e.g. microcrystalline cellulose), calcium phosphates (e.g. tricalcium phosphate, calcium hydrogen phosphatelactose) and/or titanium dioxide;
(c) lubricants such as stearic acid, calcium stearate, magnesium stearate, talc, silica, silicic acid, polyethylene glycol and/or waxes;
(d) disintegrants such as the above-mentioned starches, carboxymethyl starch, cross-linked polyvinylpyrrolidone, agar, alginic acid or a salt thereof (e.g. sodium alginate) and/or sodium starch glycollate;
(e) wetting agents such as sodium lauryl sulphate; and/or,
(f) stabilizers.

Soft gelatin capsules may be prepared with capsules containing a mixture of the Ascophyllum composition together with paraffin oil, liquid polyethylene glycols, vegetable oil, fat and/or another suitable vehicle for soft gelatin capsules. Plasticizers such as glycerol or sorbitol may also be used. Hard gelatin capsules may contain granules of the composition. Hard gelatin capsules may also contain the composition in combination with solid powdered ingredients such as those listed above.

Liquid formulations for oral administration may be prepared in the form of solutions, syrups or suspensions. Liquid formulations typically comprise the Ascophyllum composition together with an excipient such as sugar or sugar alcohols, and a carrier such as ethanol, water, glycerol, propylene glycol, polyethylene glycol, almond oil, oily esters or mixtures thereof. If desired, such liquid formulations may also contain coloring agents, flavoring agents, saccharine, thickening agents (e.g. carboxymethyl cellulose), suspending agents (e.g. sorbitol syrup, methyl cellulose, hydrogenated edible fats), emulsifying agents (e.g. lecithin, acacia), and/or preservatives (e.g. methyl p-hydroxybenzoates, propyl p-hydroxybenzoates, sorbic acid). Liquid formulations for oral administration may also be prepared in the form of a dry powder to be reconstituted with water or another suitable vehicle prior to use.

Formulations may contain one or more additional active ingredients, particularly anti-diabetic, anti-obesity, anti-oxidant or immune-stimulatory agents.

Anti-diabetic agents include e.g. insulin secretion stimulators (oral hypoglycemics), insulin sensitizers, hepatic glucose production inhibitors, and starch blockers. Insulin secretion stimulators include sulfonylureas, such as glyburide, glipizide, and glimepiride, and amino acid derivatives, such as nateglinide. Insulin sensitizers include e.g. rosiglitazone and pioglitazone. Hepatic glucose production inhibitors include e.g. metformin. Starch blockers (α-glucosidase inhibitors) include e.g. acarbose, miglitol, emiglitate, voglibose, castanospermine, and nectrisine. Various herbal products are also known to have anti-diabetic activity, including Coccinia indica, American ginseng, Gymnema sylvestre and Momordica charantia. Any of these active ingredients may be included in compositions of the invention. Mineral supplement agents, containing chromium, vanadium, and magnesium that have been demonstrated to be helpful in diabetes management may also be included.

Similarly, when used for their immunostimulatory properties, Ascophyllum compositions may contain an adjuvant to improve the immune response, or the Ascophyllum composition may itself be used as an adjuvant in various forms of mucosal vaccine preparations, especially for oral administration. Adjuvants may protect the antigen from rapid dispersal by sequestering it in a local deposit, or they may contain substances that stimulate the host to secrete factors that are chemotactic for macrophages and other components of the immune system. Known adjuvants for mucosal administration include bacterial toxins, e.g., the cholera toxin (CT), the E. coli heat-labile toxin (LT), the Clostridium difficile toxin A and the pertussis toxin (PT).

An “effective amount” of an Ascophyllum extract or composition refers to an amount effective, at dosages and for periods of time necessary, to achieve a desired prophylactic or therapeutic result, such as a reduction, inhibition, or prevention of disease onset or progression. A therapeutically effective amount may vary according to factors such as the disease state, age, sex, and weight of the individual, and the ability of the compound to elicit a desired response in the individual. Dosage regimens may be adjusted to provide the optimum therapeutic response. A therapeutically effective amount is also one in which any toxic or detrimental effects of the compound are outweighed by the therapeutically beneficial effects.

For any particular subject, specific dosage regimens may be adjusted over time according to the individual need and the professional judgement of the person administering or supervising the administration of the compositions.

For example, about 500 mg to about 10 g per day, preferably about 1-5 g per day of Ascophyllum extract or fraction would generally be administered or used on a daily basis in a human subject of average weight to obtain the desired effect.

The following examples are offered by way of illustration and not by way of limitation.

### EXAMPLE 1

This example demonstrates that highly polymeric phenolic components from Ascophyllum nodosum have antioxidant and antidiabetic properties.

### Material and Methods

Ascophyllum nodosum: Three Ascophyllum nodosum samples were used, including ON227 (collected from Nova Scotia coast, freeze-dried and milled), Asco-#40 (from Acadian Seaplants Ltd., Dartmouth, Nova Scotia, Canada), and Rockweed powder (from Maine Coast Sea Vegetables, Inc., Franklin, Me., U.S.).

Polyphenol content determination: Total phenol content of samples were measured by a modified Folin-Ciocalteu method on 96-well microplate (Singleton et al, 1999; Zhang et al, 2004).

Molecular size determination: Samples were analyzed by SEC-MALS-RI (size exclusion chromatography-multi angles laser scattering-refractive index). Sample preparation involved dissolving the samples in chloroform. The solutions were filtered using 0.2 μm sterile filters, and analyzed using Tosohaas TSK gel column (Alpha-M 7.8×300 mm, 13 μm particle size). Polystyrene was used as standard (26 KDa). HPLC operating conditions were as follows:

Flow rate: 0.6 mL/min iso-cratic
Injection volume: 100 μL
Column temperature: 35° C.
Run time: 20 min

DPPH antioxidant assay: The radical scavenging potency of samples were evaluated using DPPH (2,2-diphenyl-1-picrylhydrazyl) assay on 96-well microplate (Fukumoto & Mazza, 2000).

α-glucosidase inhibition assay: The inhibitory effect of samples on α-glucosidase was according to Sawada et al. (1993) modified to for use with enzyme extracted from rat intestinal acetone powder (Sigma I1630).

Glucose uptake assay: Glucose transport was measured in differentiated 3T3-L1 adipocytes (ATCC) using the 2-deoxyglucose method. Cells were incubated overnight in Dulbecco's minimal essential medium containing 0.2% fetal bovine serum. Cells were then incubated for 20 minutes in fresh medium without or with insulin (100 nM) and containing, or not, various concentrations of the test material. The treatment was stopped by removal of medium followed by a wash step with 1 ml of uptake buffer (phosphate buffered saline, pH 7.4, 0.5 mM MgCl₂, 1 mM CaCl₂, 2.5 mM KCl). Cells were then incubated in uptake buffer containing 50 μM deoxy-glucose (50 μM) and [³H]-deoxy-glucose (1 μCi/ml). Non-transporter-mediated 2-deoxyglucose uptake was determined in parallel in the presence of cytochalasin B (75 μM) and subtracted from both basal and stimulated uptake measurements. The uptake was stopped after 10 min. by aspiration of the uptake solution and rapidly washing the wells twice with 1 ml ice-cold 0.9% NaCl. Cells were lysed in 0.05M NaOH (1 ml/well) and 800 μl of the lysate was mixed with 3 ml of scintillation fluid and radioactivity determined. Cell protein content was determined by the Coomassie protein assay with bovine serum albumin as standard.

Oral tolerance testing: Mice (C57BL/6J strain) were made type II diabetic as described by Manchem et al. (2001). Prior to tolerance testing mice were deprived of food for 2 hours and then challenged with 2 g of maltose/kg body weight delivered via gavage (0.25 mL of a 20% solution/25 g mouse). A drop of blood was taken from the saphenous vein just prior to (0 time) and at 10, 30, 60, 120 and 180 minutes following administration of the challenge. Blood was analysed for glucose using a glucometer.

Extraction and fraction of antioxidant and antidiabetic components: ON227 (160 g) was extracted with 2 L of 50% EtOH twice under refluxing (80° C.) for 90 min. The aqueous EtOH extracts were pooled together, concentrated on Rotavap at 40° C., and then freeze-dried to yield 39.8 g of 50% EtOH extract. This extract was then suspended in 200 mL water, partitioned between EtOAc (3×300 mL), and then n-BuOH (2×300 mL). The two organic extracts were combined and dried by Rotavap to yield JK02123 (13.3 g). JK02123 was then loaded on a C-18 column and eluted sequentially with water (600 mL), water-MeOH (1:1, 800 mL), and MeOH (800 mL). The water-MeOH elute was dried to afford fraction JK02202 (5.9 g). Further fractionation of JK02202 (5.5 g) on HP-20 column eluted with water, water-MeOH (1:1), MeOH, and acetone (300 mL each) yielded fraction JK02206 (3.7 g) from the water-MeOH elute.

The alpha-glucosidase inhibition activity and polyphenol content of the aqueous EtOH extract, fractions JK02123, JK02202, and JK02206 are shown in Table 1.

TABLE 1
Alpha-glucosidase inhibition potency and total
polyphenol content of 50% aqueous EtOH extract
and purified fractions from Ascophyllum nodosum

| | | Total phenol content |
|---|---|---|
| | Alpha-glucosidase | (PGE %, phloroglucinol |
| | assay (IC50, μg/mL) | equivalent) |
| 50% EtOH extract | 77.0 | 22.5 |
| JK02123 | 38.0 | 39.8 |
| JK02202 | 24.0 | 70.2 |
| JK02206 | 16.9 | 79.0 |

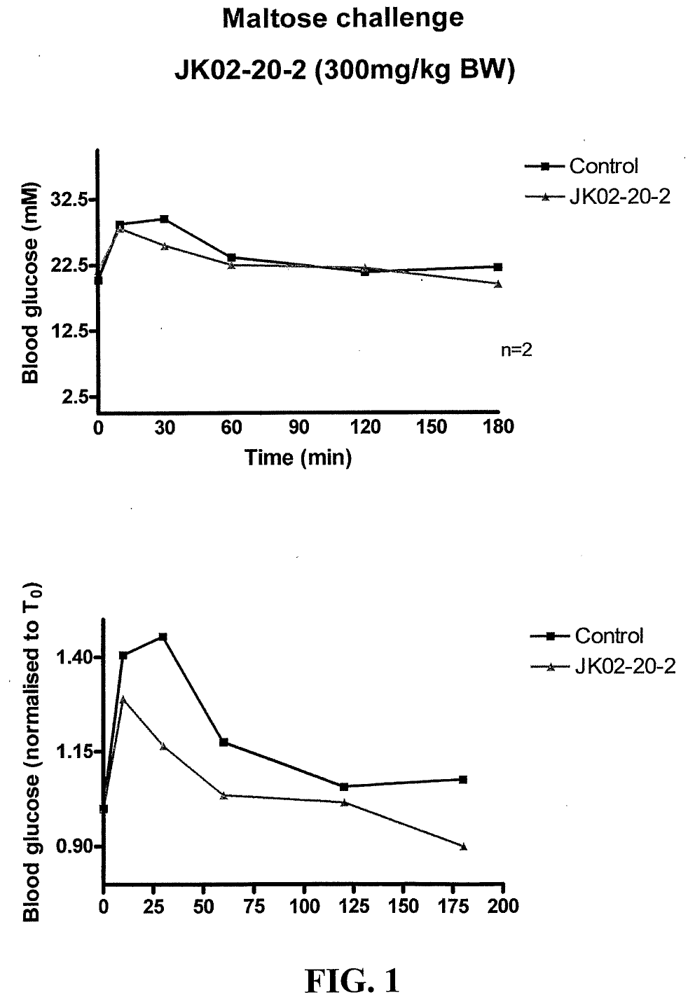

In vivo maltose challenge mice study demonstrated the blood glucose lowering effect for the purified component (JK02202, FIG. 1). Treatment of mice with JK02202 blunted the rise in blood glucose during the maltose challenge (2 g per kg body weight).

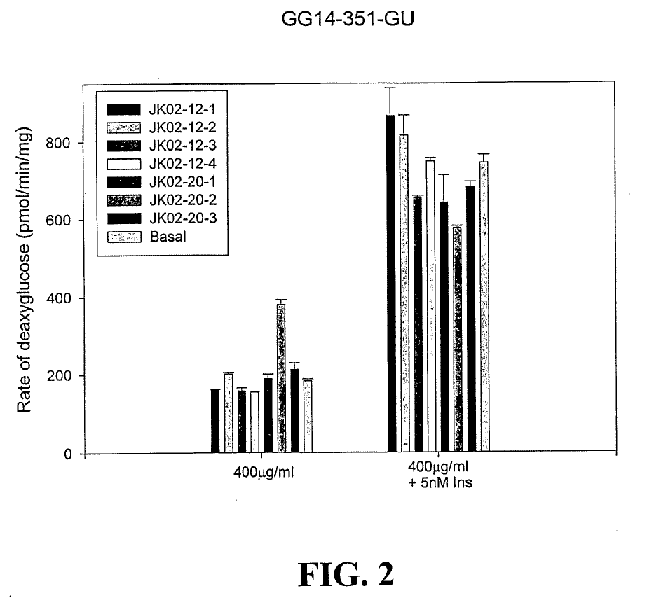

Glucose uptake assay revealed that JK02202 could induce the glucose uptake rate by about 100% in the absence of insulin (FIG. 2) which indicated its potential of insulin mimetic effect.

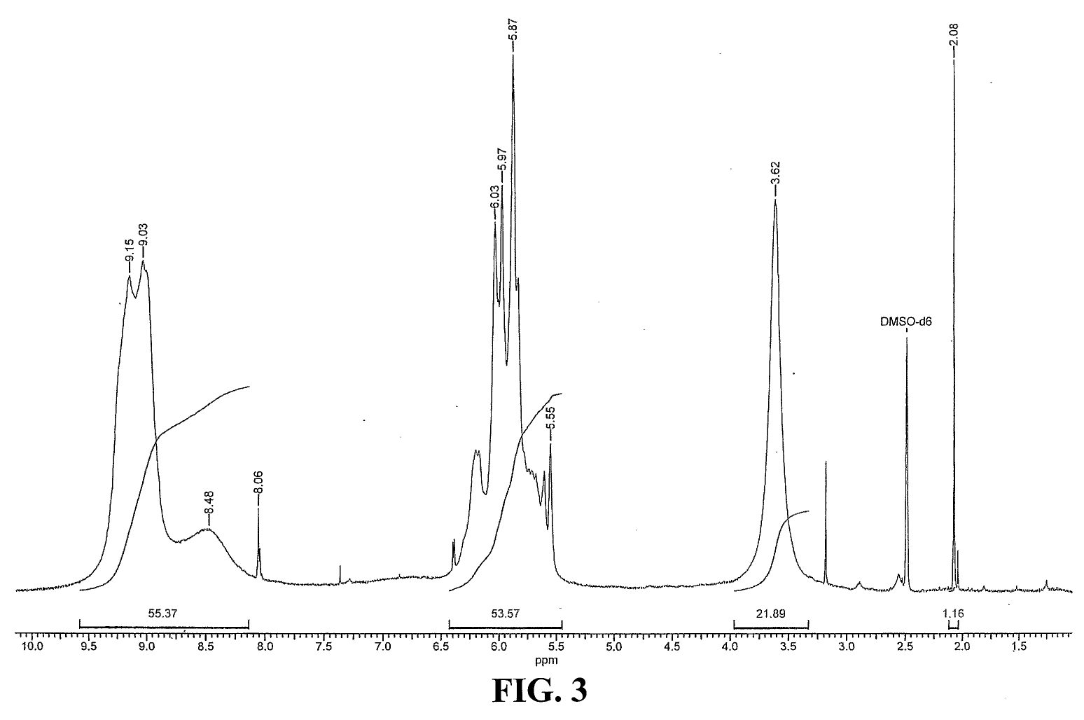

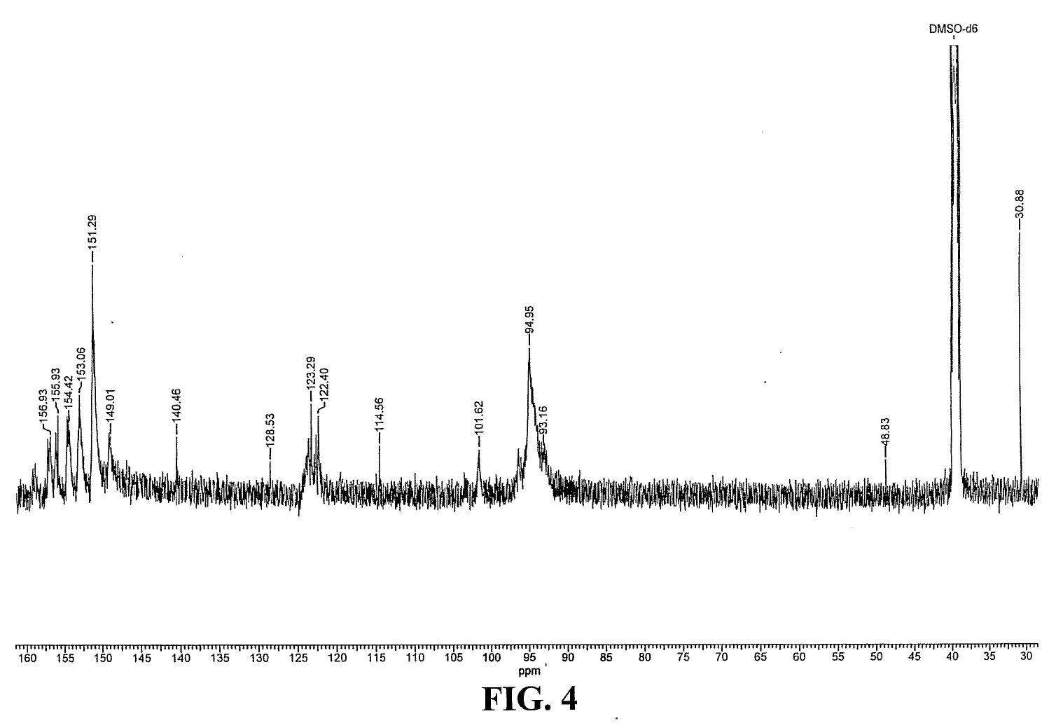

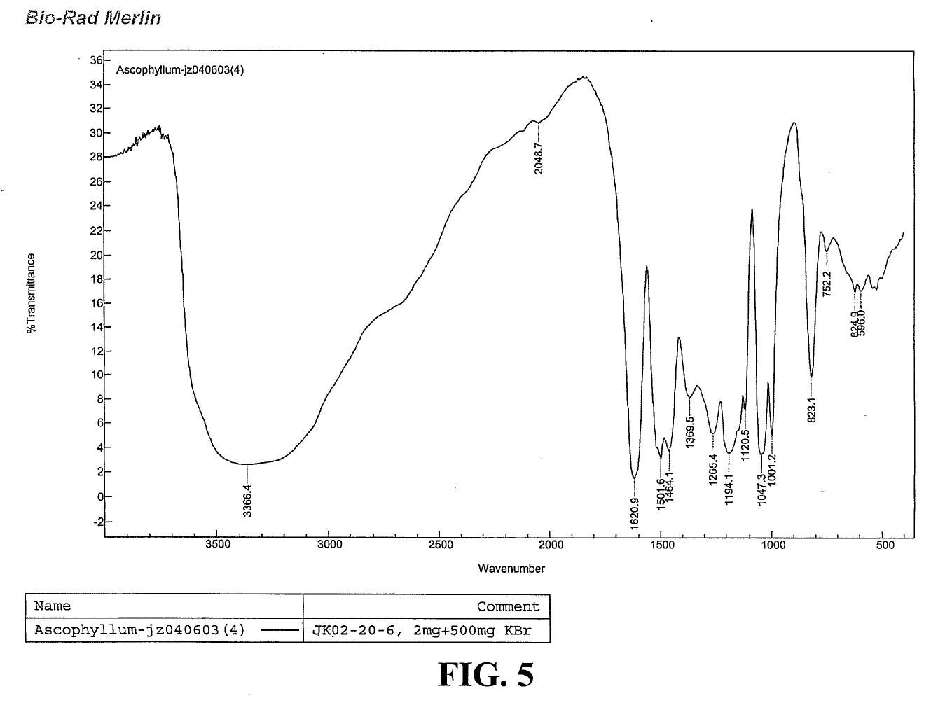

Chemical composition and structure information of the antidiabetic components obtained from Ascophyllum nodosum: ¹H- and ¹³C-NMR spectra (FIGS. 3 and 4, 500 MHz for proton and 125 MHz for carbon, in DMSO-d6) of JK02206 revealed that it is a mixture of highly polymerized phloroglucinol-based tannins (McLnnes et al, 1984). Its IR spectrum (FIG. 5) is also similar to the building block, phloroglucinol (SDBS, Integrated Spectral Data Base System for Organic Compounds, National Institute of Advanced Industrial Science and Technology, http://www.aist.go.jp/RIODB/SDBS/menu-e.html). In the proton NMR spectrum (FIG. 3), signals in the region of δ9.5-8.0 ppm are from the phenolic hydroxyl group, and the backbone aromatic proton signals are in δ6.5-5.5 ppm. The overall integration values for the two groups of distinct proton peaks are very close (55.37 for phenolic hydroxyl protons and 53.57 for phenol backbone protons), so indicates that the polymers are likely to be more linear rather than branched (in linear polymer of phloroglucinol, each unit contains two hydroxyl protons and the same number of aromatic protons; in a much branched polymer, the number of protons belongs to the two distinct types would not be the same). In carbon NMR spectrum (FIG. 4), carbons with signal in the region of δ160-145 ppm and δ125-120 ppm are oxygenated phenolic carbons. Signals at δ101.6 ppm and δ97-90 ppm can be assigned to non-oxygenated phenolic carbons; the latter one belongs to the carbons with phenolic protons. The presence of non-oxygenated carbons with the signal at δ101.6 ppm reveals that a small percentage of carbon-carbon linkage existed among the predominated C—O—C linked polymer chain.

Molecular size determination: JK02206 was methylated with dimethyl sulfate following the procedure of Glombitza & Klapperich (1985). The molecular size of this methylated product was estimated from the preliminary MALLS experiment to be in the range of 40 KDa to 1 million Dalton, with the average of 100 KDa. The result indicated the molecular size of the original phlorotannin fraction JK02206 probably in the range of 30 KDa to 830 KDa, the average MW would be 83 KDa. The degree of polymerization (DP) is thus estimated to be in the range of 200 to 8,000, with average DP of about 600.

### Experiments on Extraction Condition:

Temperature: Two samples of 80 g ON227 were extracted separately with 1.6 L 50% Aqueous EtOH at room temperature (25° C.) for 4 hrs and 80° C. (refluxing) for 1 hr twice. The aqueous EtOH extracts were concentrated on Rotavap and freeze-dried. The extraction yield, polyphenol content, and alpha-glucosidase inhibiting activity are shown in Table 2 (data for 50° C. is from the process described above)

TABLE 2
Effect of extraction temperature on the yield, polyphenol
content and alpha-glucosidase inhibition activity
of Ascophyllum nodosum components.

| | | | Alpha-glucosidase |
|---|---|---|---|
| | Extraction | Polyphenol | inhibition activity |
| | yield (%) | content (PGE %) | (IC₅₀, μg/mL) |
| 25° C. (RT) | 20.5 | 18.2 | 266 |
| 50° C. | 24.9 | 22.5 | 77 |
| 80° C. | 18.7 | 23.9 | 173 |
| (Refluxing) | | | |

Concentration of EtOH: Three milled Ascophyllum nodosum samples (20 g each) from Acadian Seaplants Ltd. (Asco-#40) and three Rockweed samples (20 g each) from Maine Coast Sea Vegetables Inc. were extracted separately with 120 mL of 30%, 50%, and 70% aqueous EtOH at 50° C. for 2 hrs. The aqueous EtOH extracts were concentrated on Rotavap and then freeze-dried. The extraction yield, total polyphenol content, alpha-glucosidase activity, and antioxidant potency are shown in Table 3.

TABLE 3
Effect of EtOH concentration on aqueous EtOH extraction of antioxidant
and antidiabetic components from Ascophyllum nodosum.

| | | | Alpha- | |
|---|---|---|---|---|
| | | | glucosidase | Antioxidant |
| | | Polyphenol | inhibition | activity |
| | Extraction | content | activity | (DPPH assay, |
| | yield (%) | (PGE %) | (IC₅₀, μg/mL) | EC₅₀, μg/mL) |
| Asco-#40 | | | | |
| 30% | 10.8 | 12.3 | 132 | 23.7 |
| 50% | 16.1 | 12.0 | 170 | 19.9 |
| 70% | 13.9 | 5.7 | 370 | 44.1 |
| Rockweed | | | | |
| 30% | 15.3 | 11.7 | 317 | 26.4 |
| 50% | 18.3 | 12.6 | 169 | 22.9 |
| 70% | 18.8 | 10.6 | 177 | 58.8 |

Experiments on Fractionation Methods:

Crude aqueous EtOH extract can be fractionated by using solvent-solvent extraction and absorbents such as C-18 and HP-20.

Fractionation using organic solvent partition: 200 g Asco-#40 powder was extracted with 1.2 L 50% aqueous EtOH at 50° C. for 2 hrs. After evaporating and drying, 32.9 g of crude extract was obtained. 20 g of this extract was suspended in 100 mL water, and partitioned between hexane (2×100 mL), EtOAc (4×100 mL), and water saturated n-BuOH (3×100 mL) sequentially. Solvents were evaporated from the EtOAc and n-BuOH soluble fractions, and yielded 1.2 g EtOAc fraction (JZ07942) and 3.6 g n-BuOH fraction (JZ07943). IR, ¹H- and

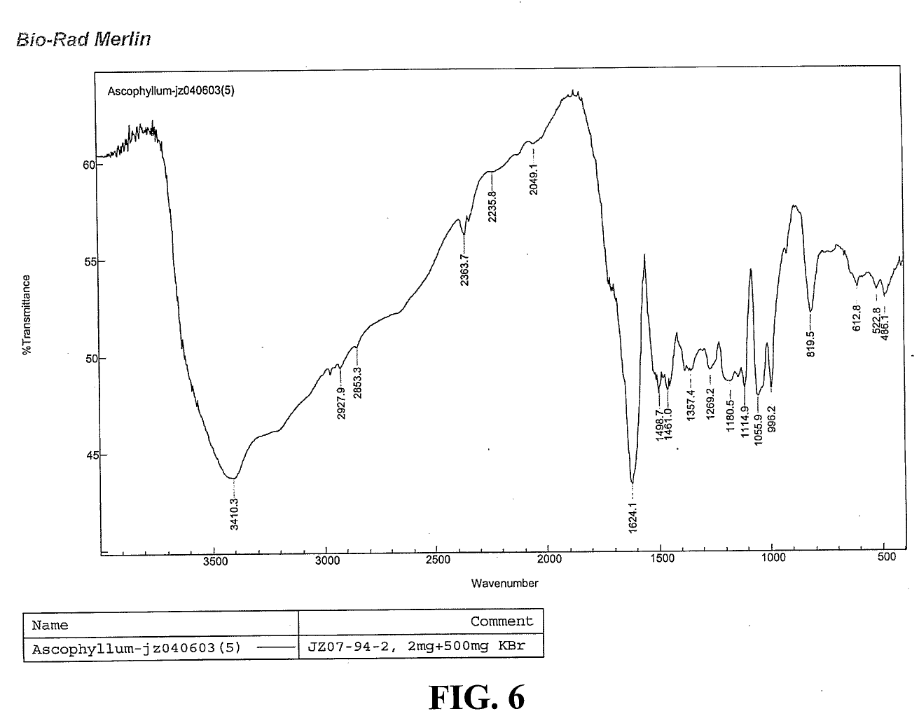

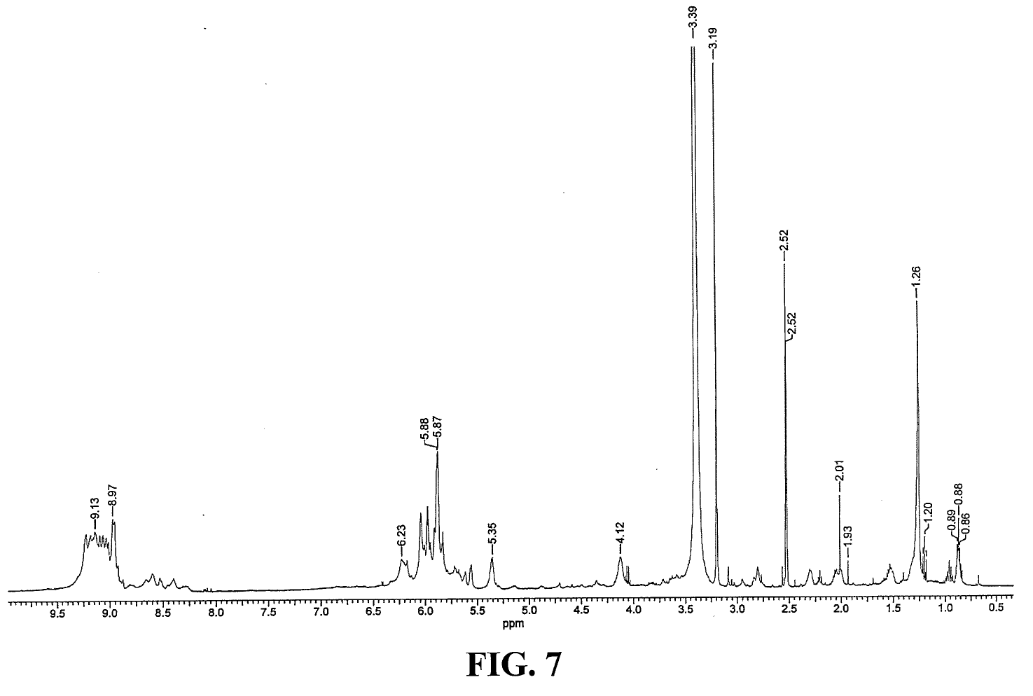

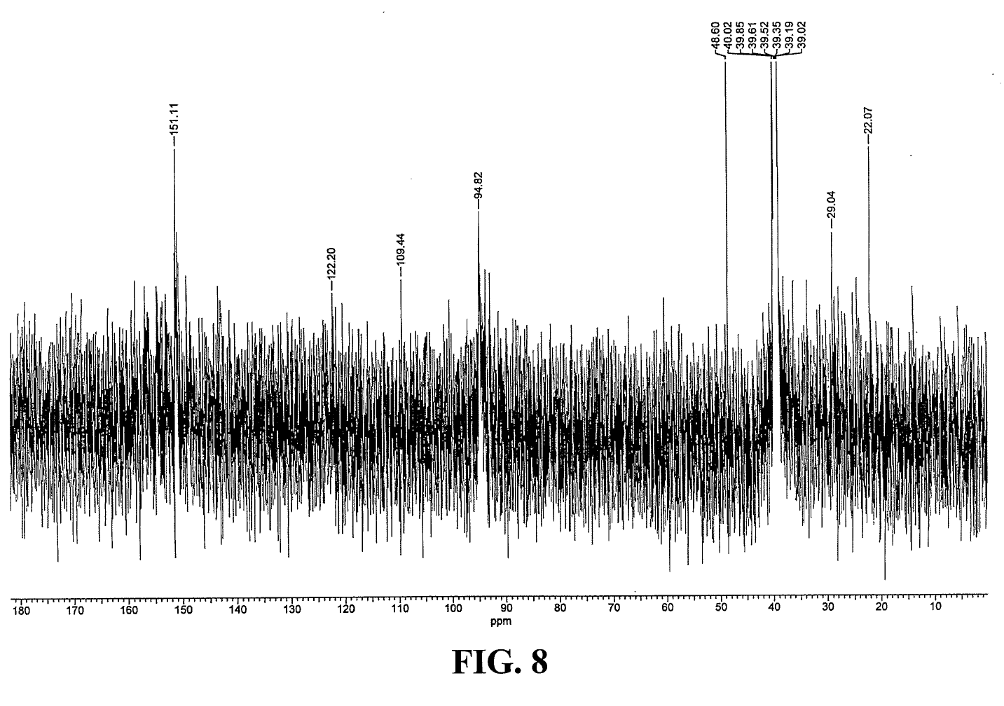

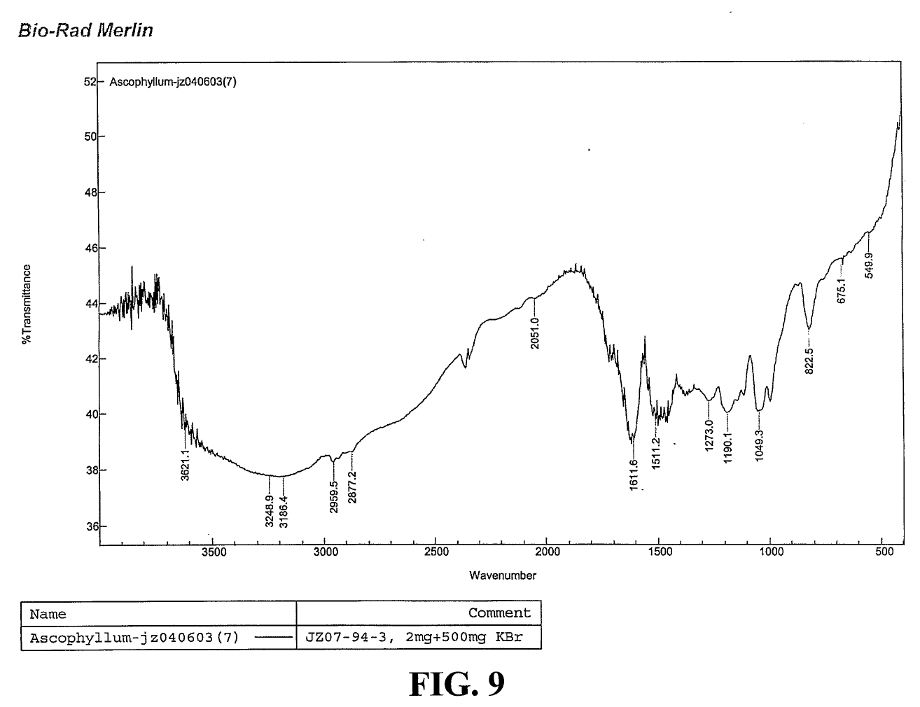

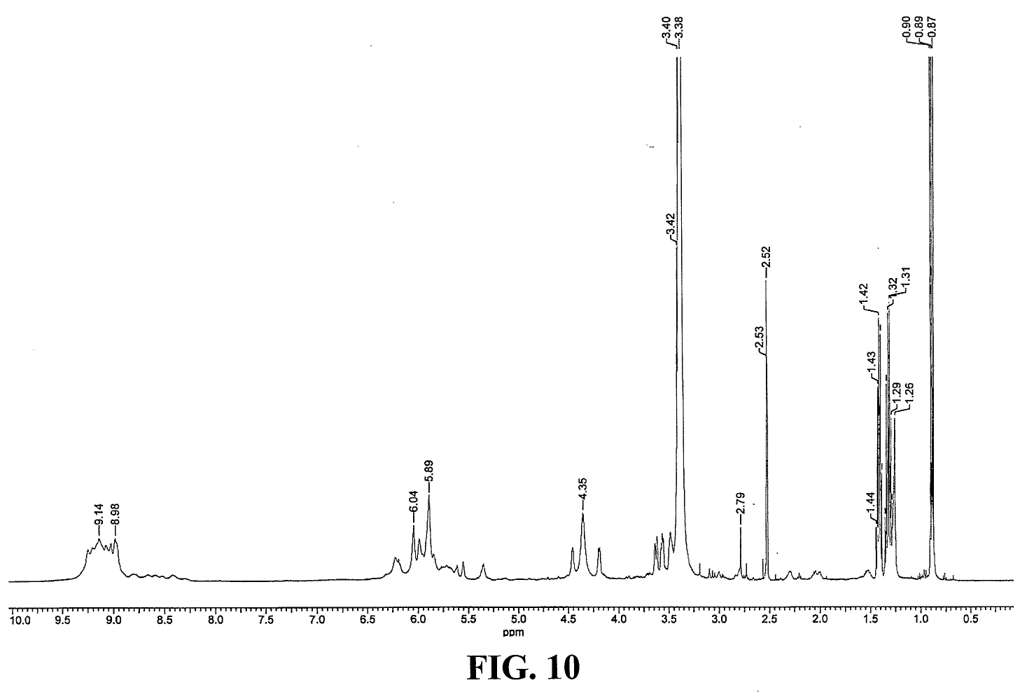

¹³C-NMR spectra (FIGS. 6, 7, and 8) of fraction JZ07942 are similar to that of fraction JZ07943 (IR and ¹H-NMR, FIGS. 9 and 10). Fraction JZ07942 appears to be more enriched in polyphenol components than JZ07943. They all have the similar compositions as JK02206. Their total polyphenol content and alpha-glucosidase inhibition activity are shown in Table 4.

TABLE 4
Total polyphenol content and alpha-glucosidase inhibition activity
of 50% EtOH extract and its organic solvent partitioned fractions
JZ07942 and JZ07943 from Ascophyllum nodosum.

| | | Alpha-glucosidase |
|---|---|---|
| | Total polyphenol | inhibition activity |
| | content (PGE %) | (IC₅₀, μg/mL) |
| 50% EtOH extract | 25.7 | 333 |
| JZ07942 | 89.6 | 55.8 |
| JZ07943 | 66.1 | 65.9 |

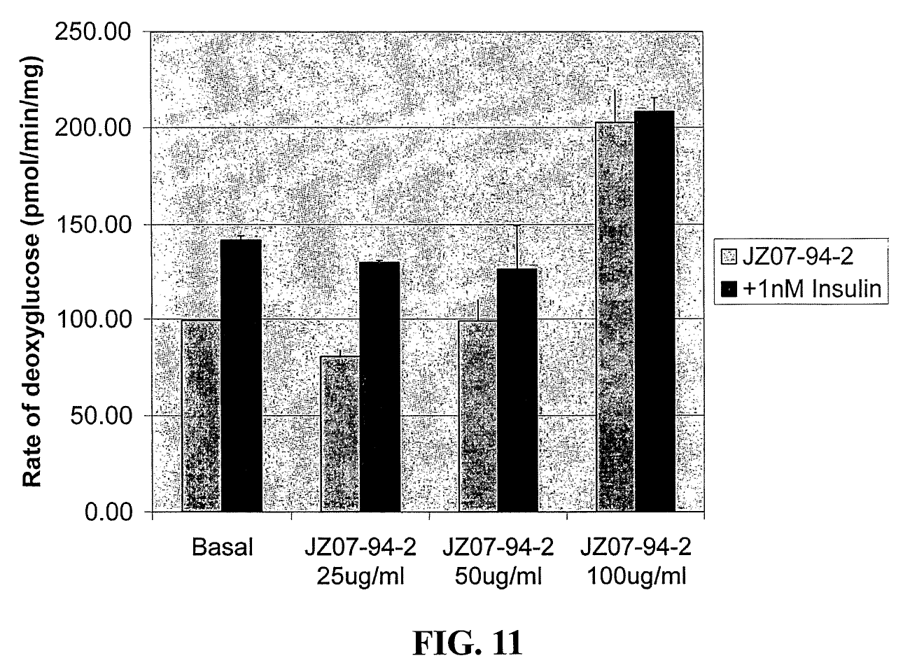

Fraction JZ07942 also showed similar insulin mimetic activity in cell-based glucose uptake assay (FIG. 11).

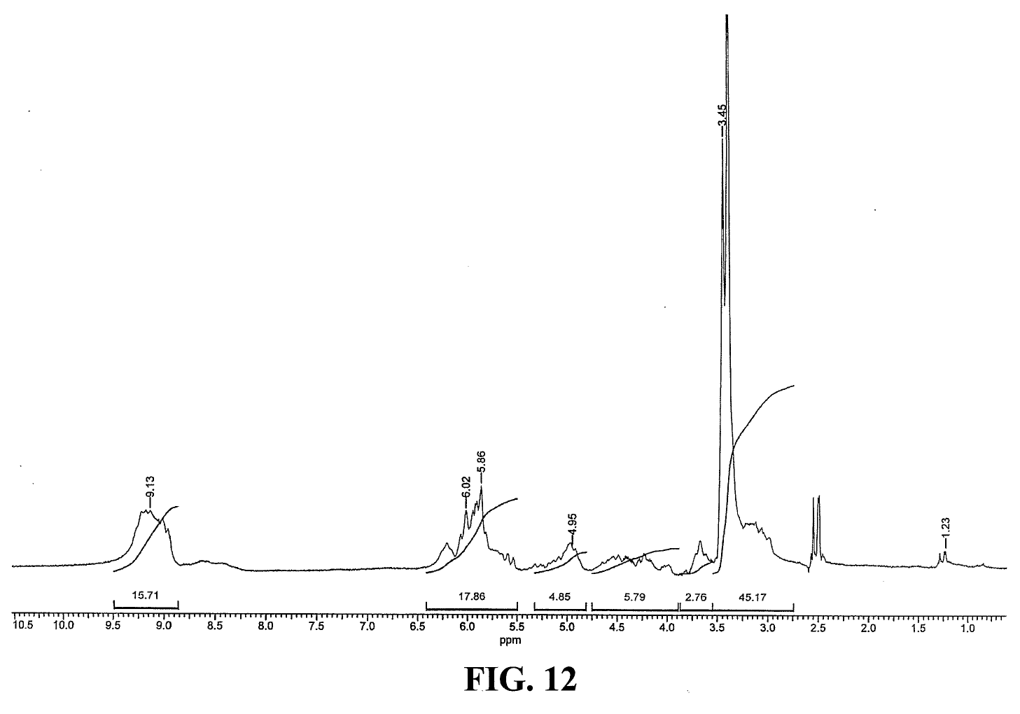

Fractionation using C-18: 20 g Asco-#40 powder was extracted with 120 mL 50% aqueous EtOH at 50° C. for 2 hrs. The liquid extract was concentrated on Rotavap and then freeze-dried to yield the 50% EtOH extract (3.2 g). Crude extract (0.9 g) was then loaded on a C-18 column, eluted sequentially with water, 95% EtOH-water (1:1), and 95% EtOH (each 200 mL). The second elute was dried to yield JK02492 (204 mg). Proton NMR of this fraction (FIG. 12) shows the composition to be similar to JK02206.

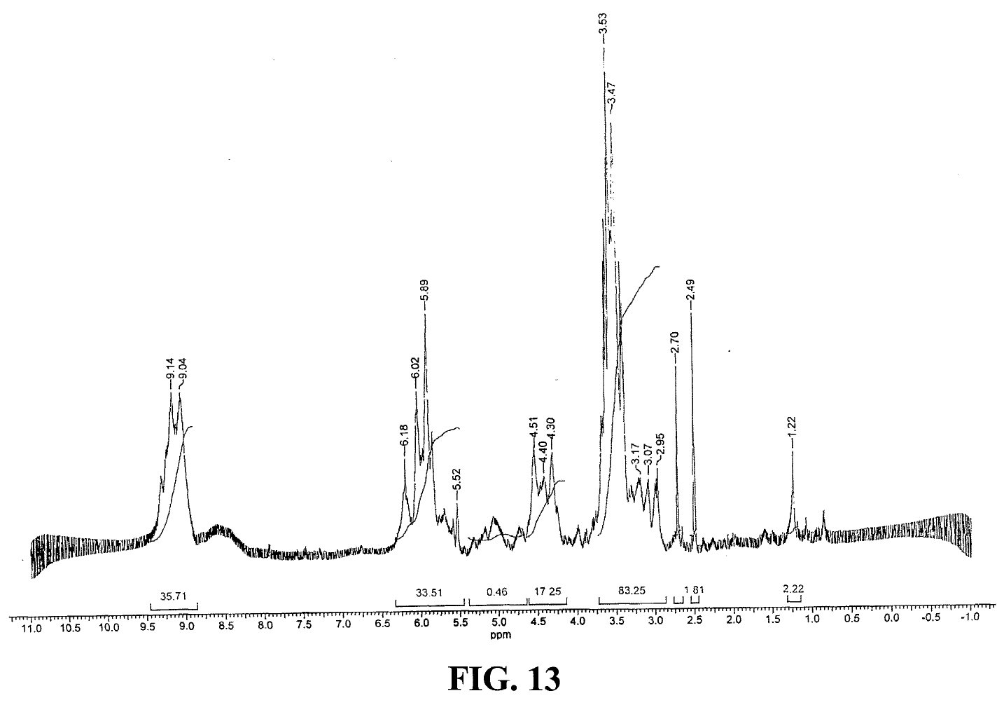

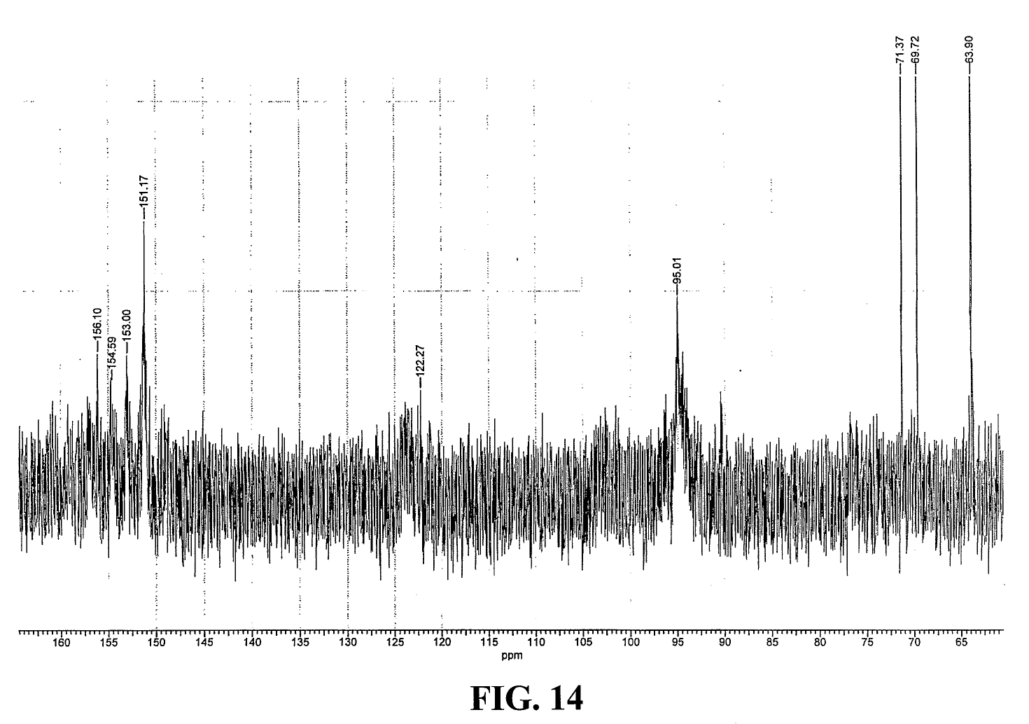

Fractionation using HP-20: 200 g Asco-#40 powder was extracted with 1.2 L 50% aqueous EtOH at 50° C. for 2 hrs. After evaporating and drying, 32.9 g of crude extract was obtained. 11.8 g of this extract was loaded on a HP-20 column, and eluted sequentially with water, MeOH:water (1:1), and MeOH (300 mL each). The MeOH-water elute was dried to afford JK02741 (2.2 g). Proton and carbon NMR of this fraction (FIGS. 13 and 14) shows the similar composition as in JK02206.

### EXAMPLE 2

This example demonstrates that sulfated polysaccharides from Ascophyllum nodosum lower blood glucose levels and have an immune-stimulating effect.

### Materials and Methods:

Ascophyllum nodosum materials: Three Ascophyllum nodosum samples were used for the investigation, including ON169 (collected from Nova Scotia coast, freeze-dried and milled), Asco-#40 powder (from Acadian Seaplants Ltd., Dartmouth, Nova Scotia, Canada), and Rockweed powder (from Maine Coast Sea Vegetables, Inc., Franklin, Me., U.S.).

Determination of sulfated polysaccharides content: A 96-well microplate method (Zhang et al, 2004). Briefly, 1˜2 mg of sample was dissolved in water, centrifuged and then diluted to prepare a final sample solution with a concentration of 100˜200 μg/mL. 20 μL of this sample solution was loaded on the microplate, mixed with 100 μL dilute HCl (pH 1.5), and 80 μL Methylene Blue (MB) solution (43.5 μg/mL). The absorbance at 660 nm was recorded and the sulfated polysaccharides content of the testing sample was calculated from the value of sample related to the calibration curve of standard substance fucoidan (from Aldrich), and described as percentage of fucoidan equivalent (FUE %).

Level of sulfation determination: AOAC Official Method 973.57 (turbidimetric method).

Molecular size determination: Samples were analyzed by SEC-MALS-RI (size exclusion chromatography-multi angles laser scattering-refractive index). Sample preparation involved dissolving the samples in 88 mM sodium acetate buffer (pH 4.5) containing 40 ppm sodium azide, to a concentration of ˜2.5 mg/mL. The solutions were filtered using 0.2 μm sterile filters, and analyzed using two size exclusion columns in series (Tosohaas TSK gel G3000PWxl and TSK gel G2500PWxl 7.8×300 mm, 6 μm particle size) with a guard column (PWXL 4 cm×6 mm). LC and RI operating conditions were as follows:

Flow rate: 0.6 mL/min iso-cratic
Injection volume: 100 μL
Temperature of sample tray: 10° C.
Run time: 45 min
RI temperature: 35° C.

Monosaccharide composition analysis: A standard alditol acetate derivation and GC-MS analysis was used. 2˜4 mg of sample in duplicate was mixed with 1 mL of 1 M TFA (trifluoroacetic acid), sealed and heated at 100° C. for 3 hrs. The mixture was evaporated and then added sodium borohydride solution to stir overnight. Evaporated again to dryness using the nitrogen evaporator, added acetic anhydride and heated at 80° C. for 2 hrs. The product was dissolved in EtOAc and analyzed by GC-MS.

Blood glucose testing: Mice (C57BL/6J strain) were deprived of food for 2 hours and then injected intraperitoneally with test substance (dissolved in phosphate-buffered saline, PBS) or PBS. A drop of blood was taken from the saphenous vein just prior to (0 time) and at 0.5, 1, 2, 4 and 6 hours following the injection. Blood was analysed for glucose using a glucometer.

Macrophage activation assay: RAW264.7 macrophage cells were cultured in Dulbecco's minimal essential medium supplemented with 10% heat-inactivated fetal bovine serum, 1 mM pyruvic acid, 4 mM glutamine, and 1% penicillin-streptomycin (100 U/ml each) at 37° C. in 5% CO₂.

Cells were seeded in 48-well plates at a density of 2.5×10⁵ cells per well in 500 μL culture medium (without phenol red) and grown overnight to 80-90% confluency. Samples were dissolved in culture medium at 375 μg/mL and passed through a sterile, 0.22 μm filter. One hundred μL of an extract stock solution, or an appropriate dilution, was added to the triplicate wells (final volume 600 μL). Control wells received 100 μL of culture medium alone. LPS (0.5 μg/mL) was routinely run as a positive control. Experiments were performed in duplicate or triplicate and, after 24 h treatment, culture medium from replicate wells were pooled and assayed for nitrite concentration. This method was adapted from procedures described by Reninger et al. (2000).

Nitric oxide production was assessed by measuring nitrite concentration in 50 μL of cell culture medium. Samples were incubated with 50 μL Griess Reagent (1.0% Sulfanilamide, 0.10% N-(1-Naphthyl)ethylenediamine dihydrochloride and 2.5% Phosphoric Acid) for 5 minutes at room temperature and absorbance measured at 550 nm. Sodium nitrite dissolved in culture medium was used as standard.

Preparation of Blood Glucose Lowering and Immune Stimulating Extracts and Fractions from Ascophyllum nodosum:

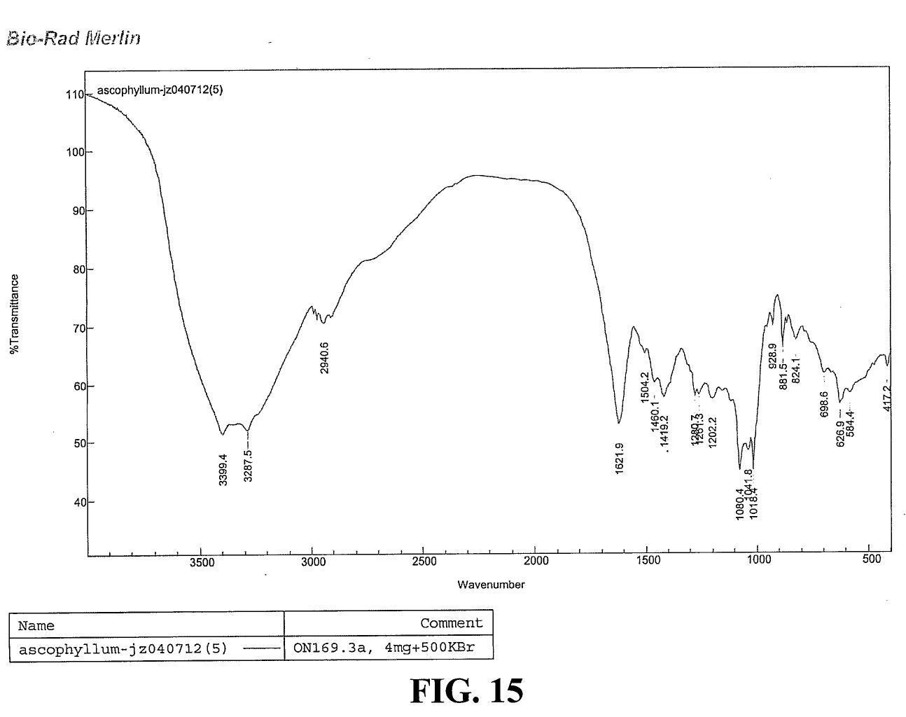

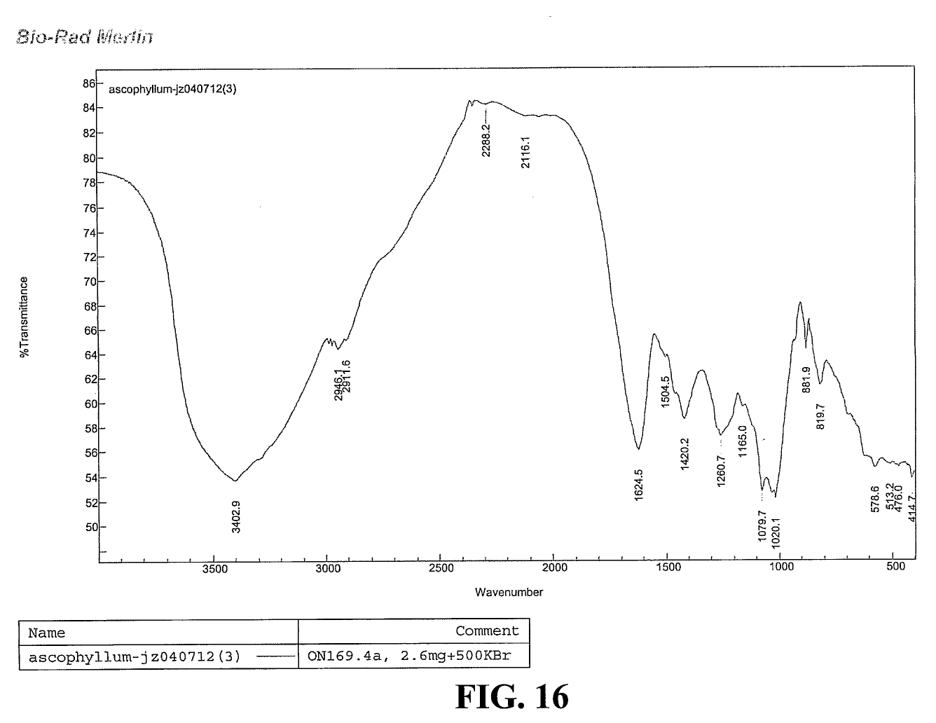

50 g ON169 powder was extracted with hexane (2×300 mL) and MeOH (2×300 mL) for 24 hrs to remove lipophilic components. The seaweed residue was then sequentially extracted with water at room temperature (300 mL, 24 hrs) and then at boiling (300 mL, 2 hrs). The aqueous extracts were then freeze-dried to yield ON169.3a (RT, 3.3 g) and ON169.4a (boiling, 2.6 g). The IR spectra of the two water extracts (FIGS. 15 and 16) are similar, indicating a similar composition for these two samples.

1.6 g ON169.4a was further fractionated by precipitating sequentially in 50% and 80% EtOH to obtain fractions JZ07311 (50% EtOH precipitate, 473 mg), JZ07312 (80% EtOH precipitation, 35 mg), and JZ07313 (80% EtOH soluble, 891 mg). The sulfated polysaccharide contents for the hot water extract and its fractions are listed in Table 5.

TABLE 5
Sulfated polysaccharide contents of ON169.4a and its fractions
JZ07311, JZ07312, and JZ07313 from Ascophyllum nodosum

| | Sulfated polysaccharide content (PUE %) |
|---|---|
| ON169.4a | 20.7 |
| JZ07311 | 41.4 |
| JZ07312 | 47.3 |
| JZ07313 | 9.3 |

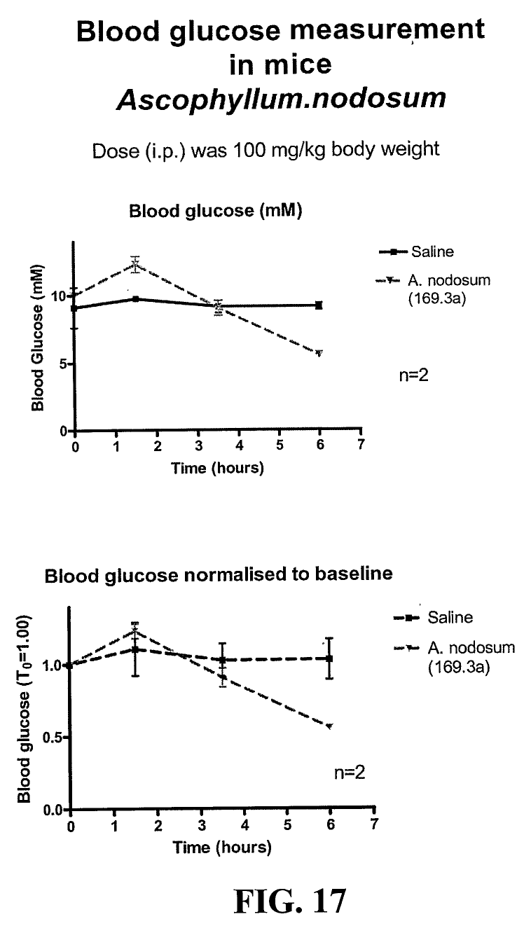

Blood glucose lowering effects of ON169.3a were observed in diabetic mice study, as shown in FIG. 17.

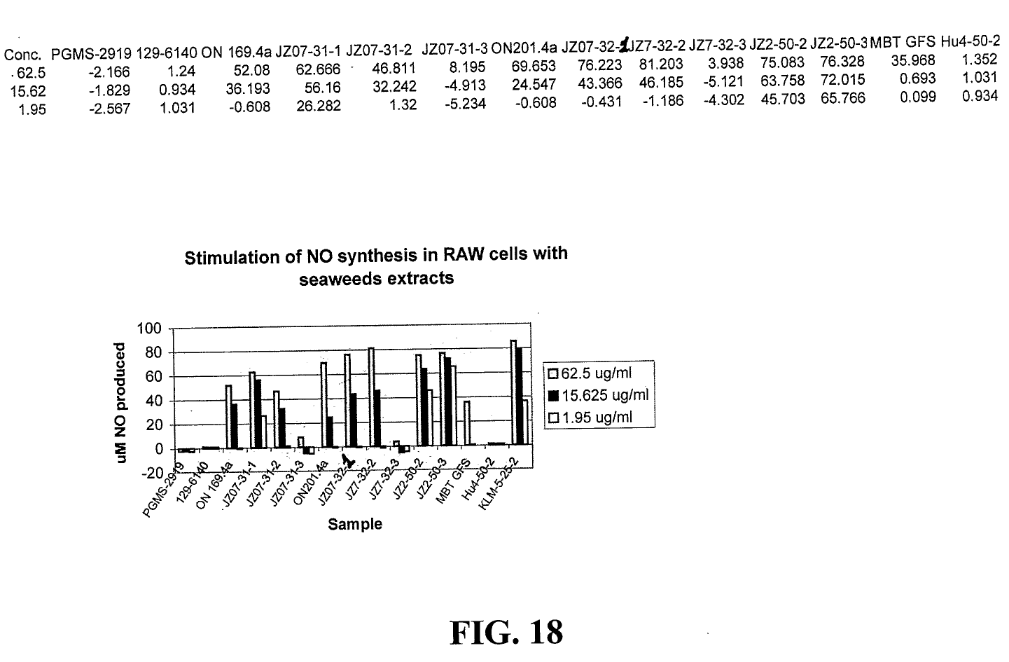

ON169.4a and its fractions JZ07311 and JZ07312 were shown to have immune stimulation activity in our macrophage assay (FIG. 18).

### Effects of Temperature on the Yield of Extraction:

Three Asco-#40 and three Rockweed powder samples (20 g each) were extracted with 200 mL water at room temperature (RT), 50° C., and 80° C. for 2 hrs. The extracts were freeze-dried; yield for each of the extractions is shown in Table 6.

TABLE 6
Effects of temperature on the extraction yield of
bioactive components from Ascophyllum nodosum

| | Asco-#40 (%) | Rockweed (%) |
|---|---|---|
| RT | 11.9 | 14.6 |
| 50° C. | 11.8 | 15.7 |
| 80° C. | 14.5 | 18.8 |

### Experiments on Fractionation Methods:

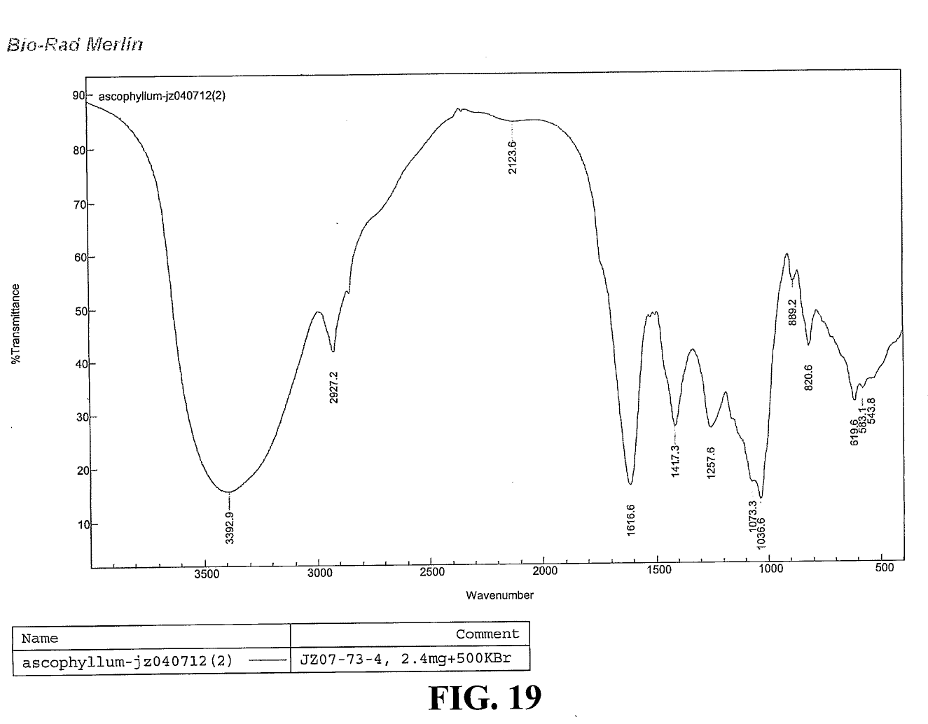

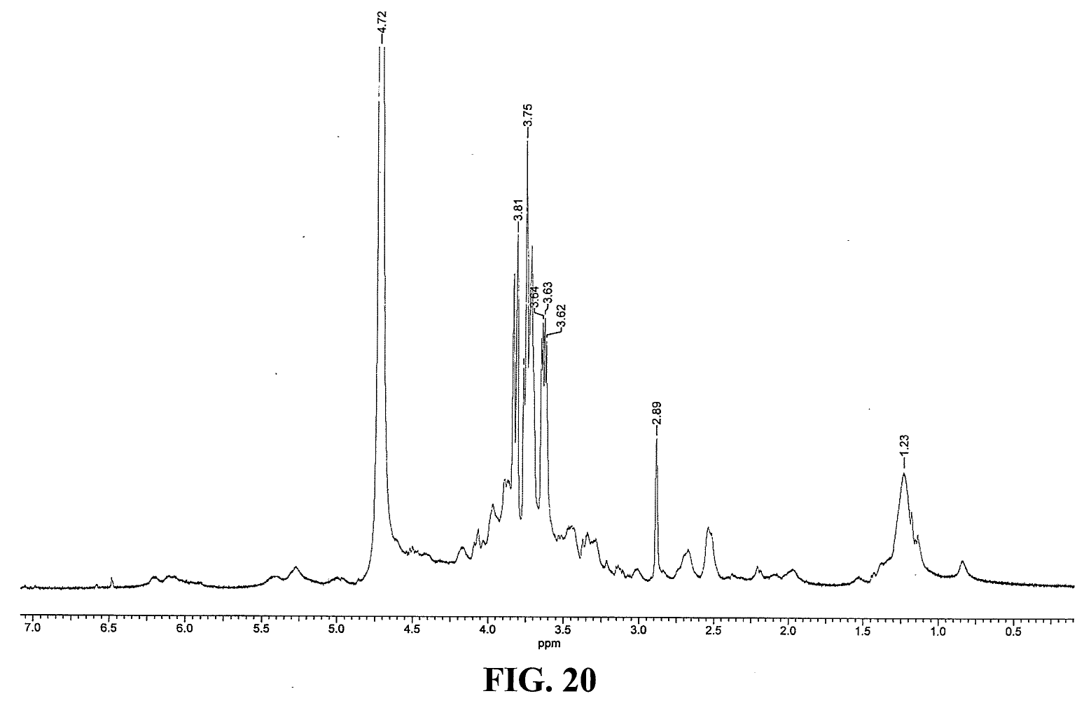

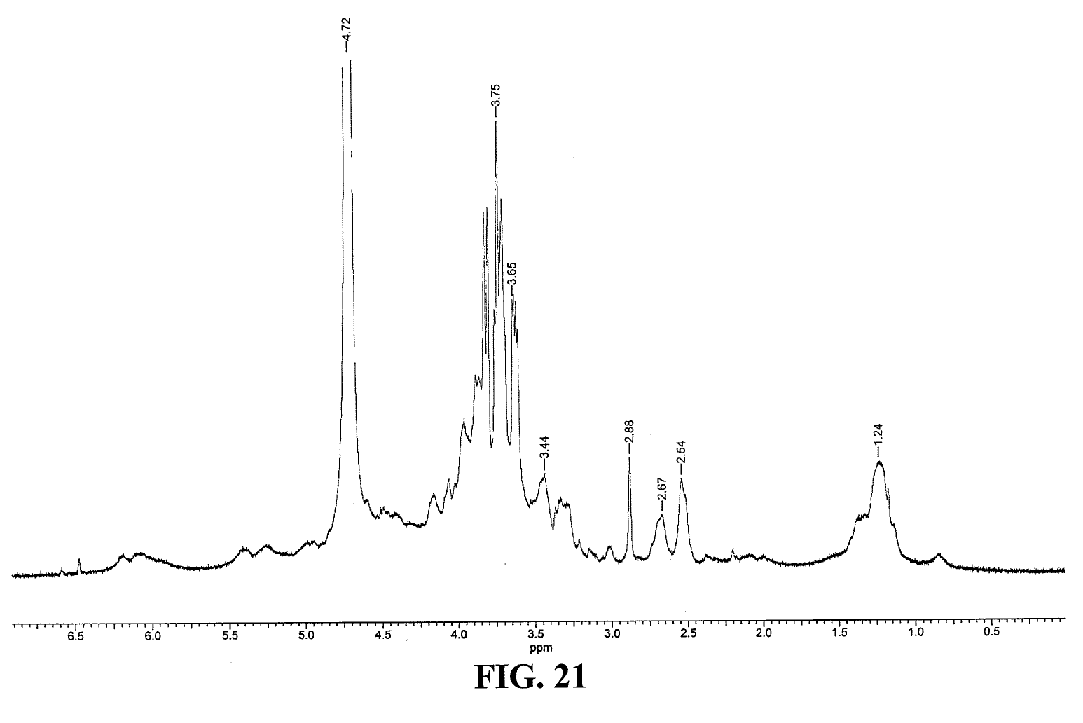

Ultrafiltration: 20 g Asco-#40 was extracted with 120 mL of 50% EtOH at 50° C. for 2 hrs to remove lipophilic components. The seaweed residue was then extracted with 200 mL water at 80° C. for 2 hrs, then freeze-dried to yield a hot water extract JZ07734 (1.1 g). 202 mg of this extract was dissolved in 10 mL water, after centrifugation, the supernatant was introduced into an ultrafiltration system (membrane MW cut-off 10 KDa). Fraction retained by the membrane was freeze-dried to yield JZ07892 (141 mg). IR and proton NMR spectra of JZ07734 (FIGS. 19 and 20) and proton NMR spectrum of JZ07892 (FIG. 21) are attached, the NMR spectra of the them indicate that ultrafiltration can be used as a fractionation approach to enrich the polysaccharide components.

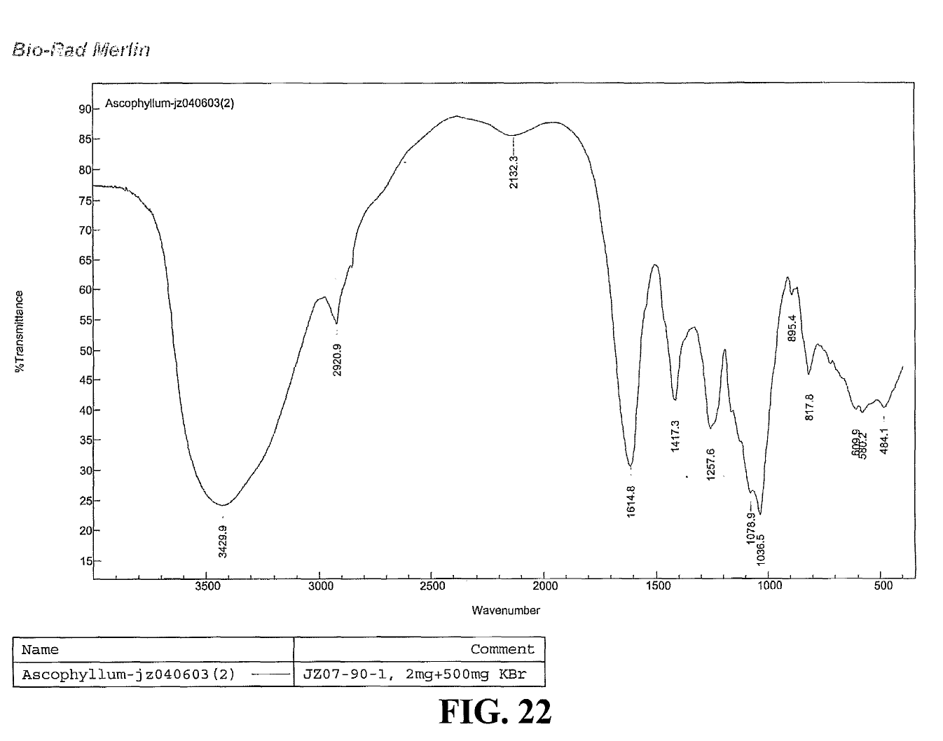

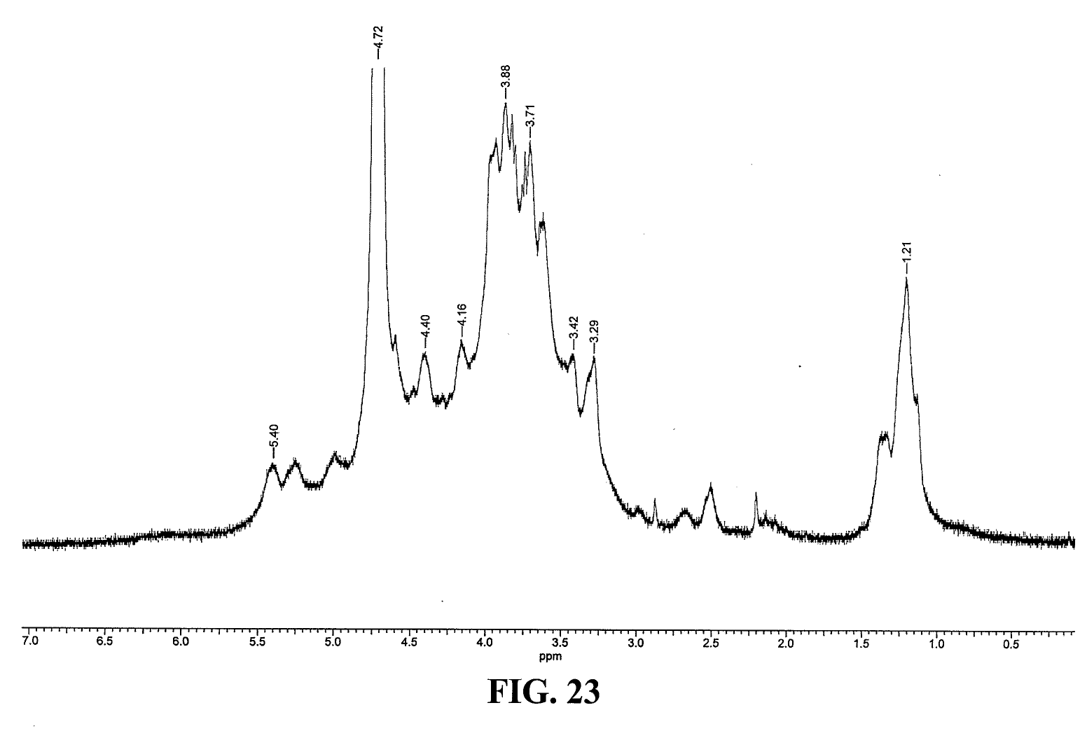

EtOH precipitation: 407 mg Asco-#40 hot water extract JZ07734 was dissolved in 20 mL water, after centrifugation, 60 mL 95% EtOH was added while stirring. The precipitate was collected by centrifugation and freeze-dried to afford fraction JZ07901 (146 mg). IR and proton NMR of this EtOH precipitate are shown as FIGS. 22 and 23. EtOH precipitation was revealed to be an effective method to obtain a polysaccharide-enriched fraction.

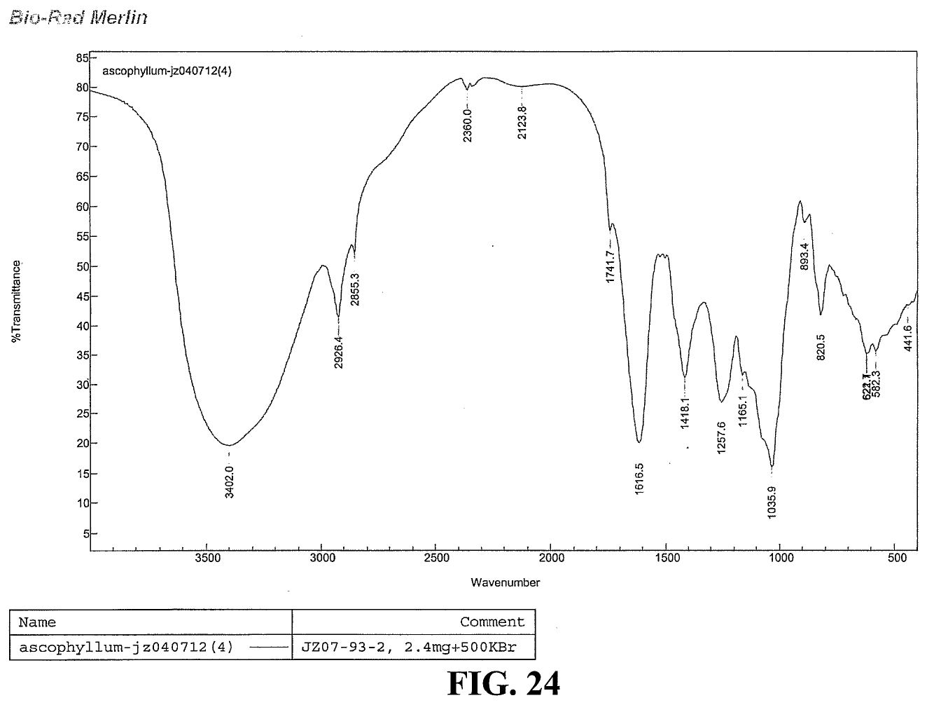

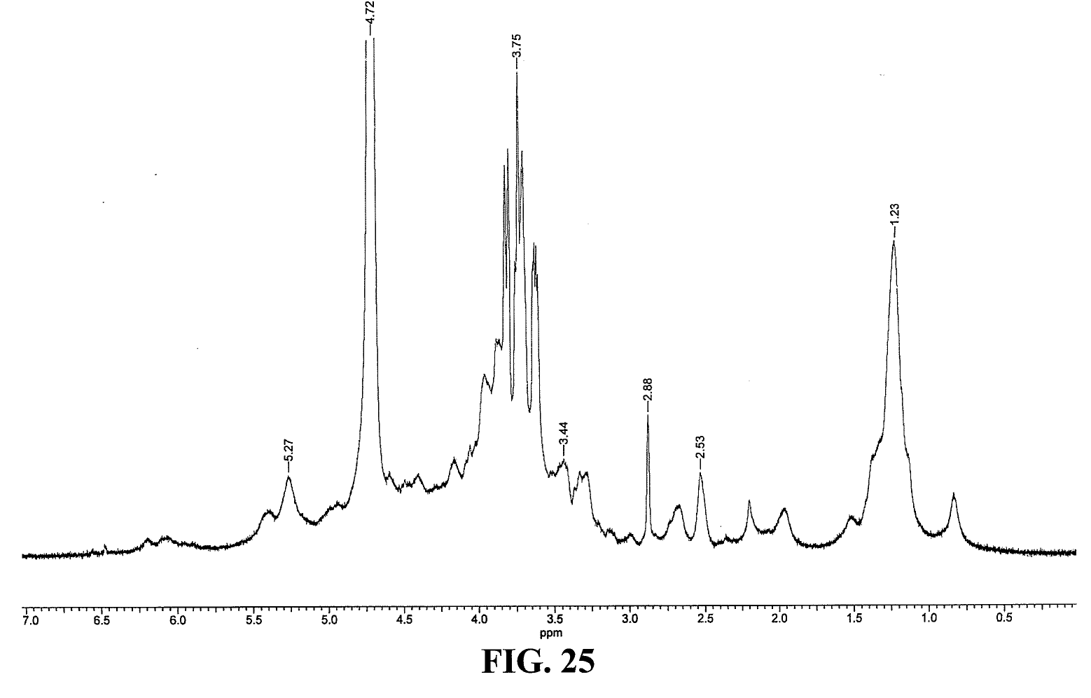

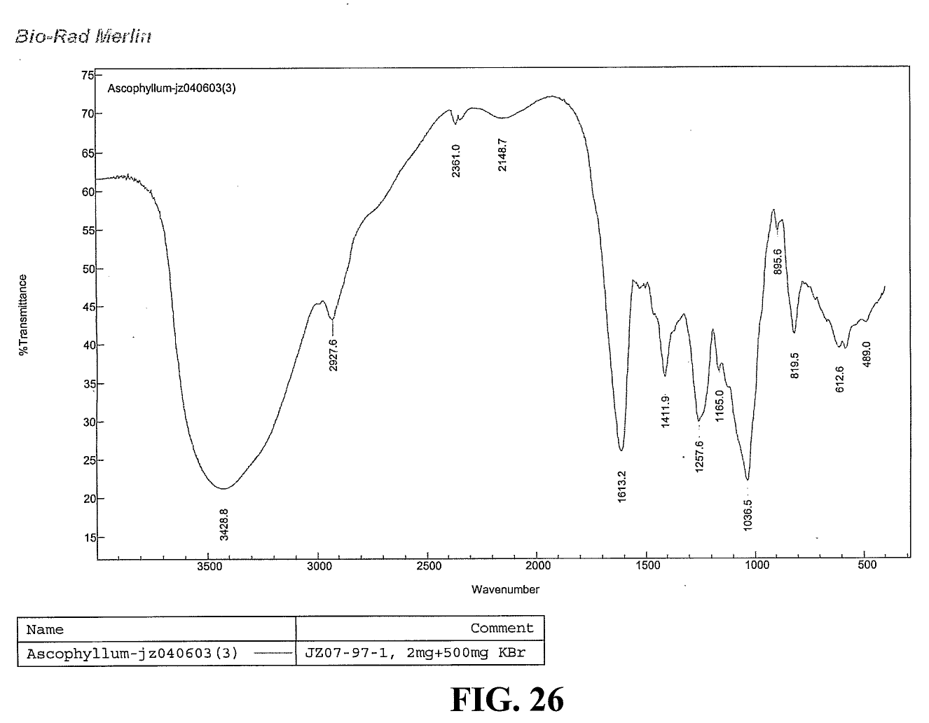

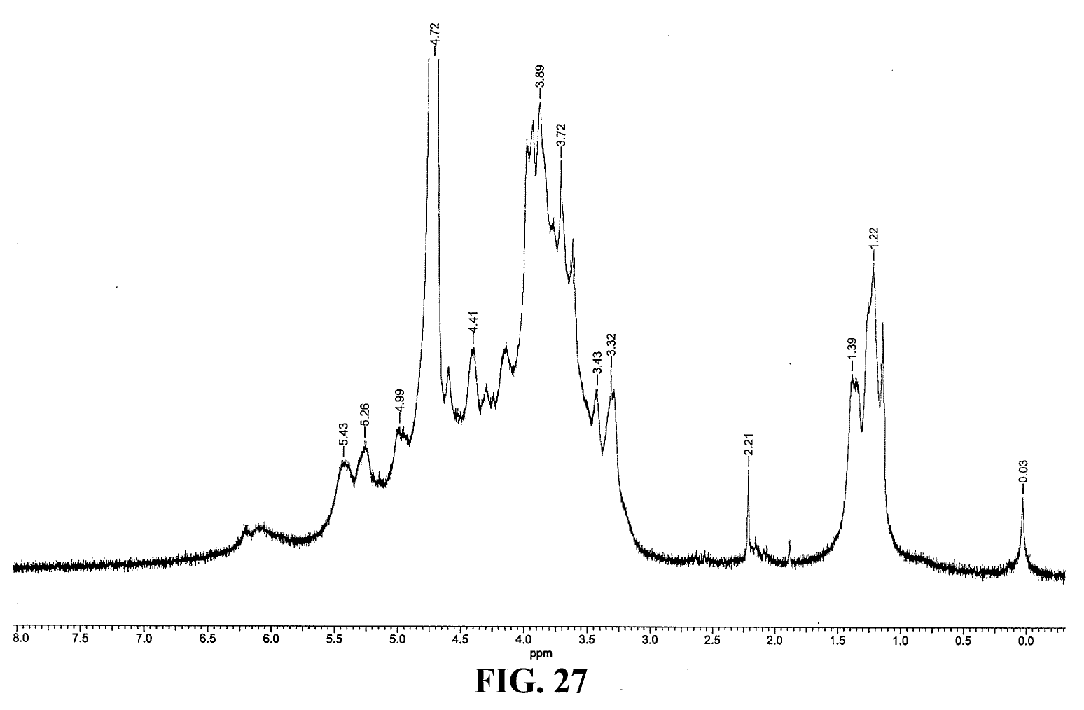

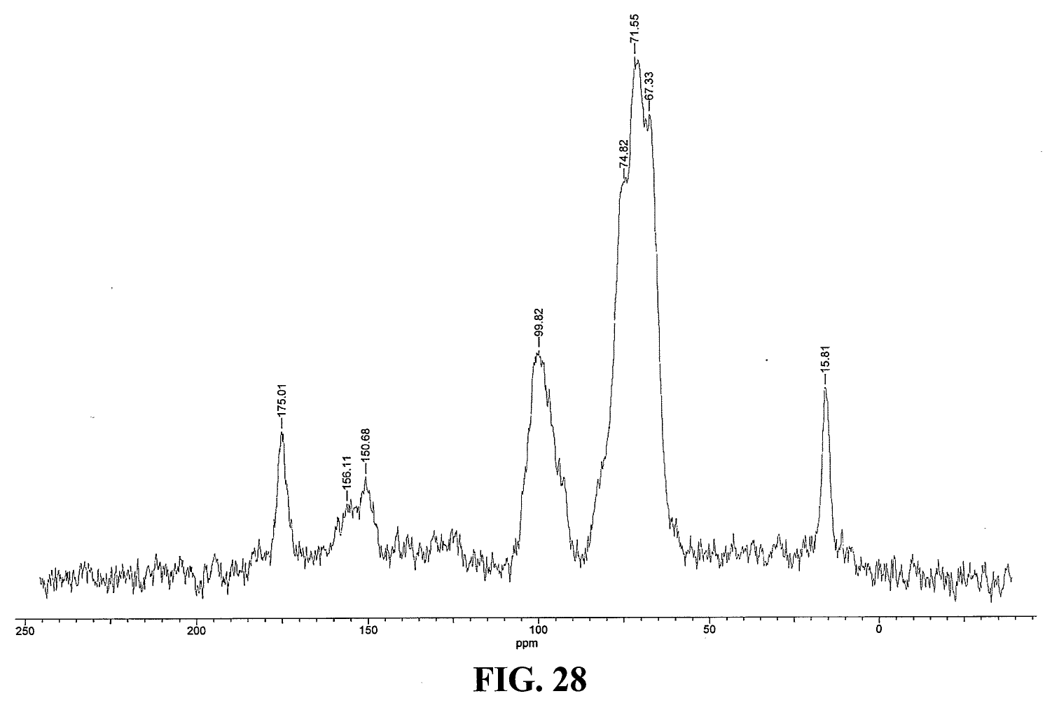

CTAB (cetyltrimethylammonium bromide) precipitation: 200 g Asco-#40 was extracted with 1.2 L 50% EtOH at 50° C. for 2 hrs to remove the lipophilic components. The seaweed residue was then extracted with 2 L water at 80° C. for 2 hrs, and a hot water extract JZ07932 (17.8 g) was obtained after freeze-drying. 285 mg of this hot water extract was dissolved in 30 mL 0.01 M Na₂SO₄ solution, then added 100 mL 1% CTAB solution. The precipitate was collected by centrifugation, and then washed sequentially with KCl saturated EtOH, KCl saturated 95% EtOH, and then 95% EtOH. The residue was dissolved in water and freeze-dried to yield JZ07971 (148 mg). IR and proton NMR spectra of JZ07932 and JZ07971, as well as a solid phase carbon NMR spectrum of JZ07971 are shown as FIGS. 24-28. CTAB precipitation was revealed to be an effective method to obtain a polysaccharide-enriched fraction.

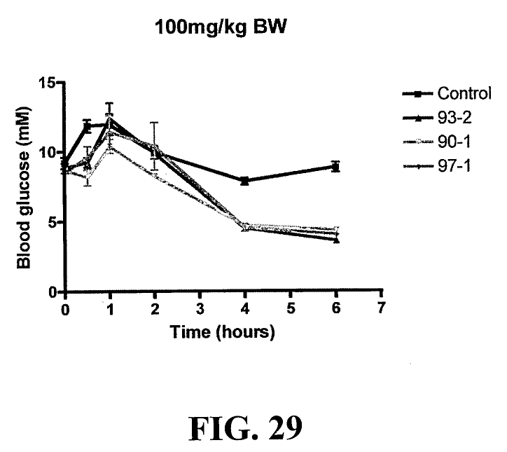

Blood glucose lowering effects of JZ07932, JZ07901 and JZ07971 in mice is shown in FIG. 29.

The sulfated polysaccharides content and level of sulfation of polysaccharide-enriched fractions JZ07892, JZ07901, and JZ07971 are listed in Table 7.

TABLE 7
The sulfated polysaccharide content and level of sulfation of
polysaccharide-enriched fractions from Ascophyllum nodosum

| | Sulfated | |
|---|---|---|
| | polysaccharide | Level of sulfation |
| | content (PUE %) | (% sulfate) |
| JZ07892 | ND* | 10.0 |
| JZ07901 | 49.2 | 12.2 |
| JZ07971 | 70.8 | 13.2 |

ND: not determined.

Molecular size of fractions JZ07901 and JZ07971: The molecular size of JZ07901 and JZ07971 was revealed by SEC-MALS analysis to be bigger than 100 KDa. The average MW for JZ07901 is 520 KDa, while for JZ07971 is 853 KDa.

Monosaccharide compositions of JZ07901 and JZ07971: see Table 8.

TABLE 8
Monosaccharide compositions of polysaccharide-enriched
fractions JZ07901 and JZ07971 from Ascophyllum nodosum

| | JZ07901 | JZ07971 |
|---|---|---|
| Fucose | 37.5% | 46.0% |
| Xylose | 29.5% | 28.0% |
| Mannose | 13.0% | 10.0% |
| Galactose | 8.5% | 8.5% |
| Glucose | 7.5% | 4.0% |
| Unknown | 4.0% | 3.5% |

Structure Characteristics of Blood Glucose Lowering and Immune Stimulating Components from Ascophyllum nodosum:

Without being bound to a particular theory, we believe that the sulfated polysaccharides from Ascophyllum nodosum are the bioactive components for blood glucose lowering and immune stimulation activities. The sulfated polysaccharide enriched fractions would have a sulfation level of 5-30%, and sulfated polysaccharide content of 40-100%. The molecular size of the enriched fractions are from about 100 kDa to about 3000 kDa, with average molecular weight in the range of about 300 to about 1000 kDa. The monosaccharide compositions for these polysaccharide components were shown to be primarily Fucose and Xylose, with less amount of mannose, galactose, and glucose.

### EXAMPLE 3

This example demonstrates the antidiabetic efficacy of Ascophyllum nodosum fractions in vivo. This study involved a 4-week treatment experiment with streptozotocin-induced diabetic mice (Experiment 1), a glucose challenge experiment on diabetic mice (Experiment 2), and a sucrose challenge experiment on diabetic mice (Experiment 3).

### Study Design and Protocol

### Experiment 1 (4-Week Treatment Study)

Animal Model. Male mice (18-20 g) of the KunMing strain from Hebei Experimental Animal Center were kept in the facility for 2 days, before starting the experiment. Diabetes was induced in mice with a single injection of streptozotocin (STZ). After fasting for 12 hrs, male mice with body weight above 20 g were given STZ dissolved in sodium citrate buffer via tail vein at a dose of 110 mg/kg body weight. After 8 days, diabetic mice with blood glucose levels between 11 to 20 mmol/L were entered in the study. Diabetic animals were divided into 5 groups (10 mice per group). Of these, 3 groups were treated with test samples, one group was a control group, receiving vehicle alone, and one group received metformin, an oral anti-diabetic agent. A sixth group, consisting of non-diabetic mice, was also included for comparison. They were treated with vehicle alone. Mice were housed 5 to a cage with free access to food and water.

Test Samples and Dose. A. nodosum samples tested were as follows:

1) crude extract, CE
2) polyphenolic fraction, PPF
3) polysaccharide fraction, PSF

Samples were dissolved in carboxymethylcellulose (CMC; 0.5%). Test sample solutions were prepared every morning at a concentration of 20 mg/ml in distilled water and given via gavage at a volume of 0.2 mL/20 g BW to deliver a dose of 200 mg/kg body weight. The control groups were gavaged with distilled water (0.2 mL/20 g BW). Metformin was gavaged at a dose of 250 mg/kg in 0.2 ml distilled water. Animals were treated daily for 4 weeks.

Measurements. Animals were monitored as follows:
1) General activity was monitored on a daily basis.
2) Body weight was measured weekly.
3) Water and food consumption were measured weekly.
4) Fasting blood glucose level was measured on Day 0, Day 7, Day 14, and Day 28 (blood from tail vein).
5) On Day 28, blood was collected from the orbital vein for blood antioxidant activity, serum total cholesterol, triglycerides, and glycated hemoglobin determinations.
6) On day 28, animals were killed and liver removed for analysis of glycogen content.

### Experiment 2 (Glucose Challenge)

Animal Model and Treatment. Animals were made diabetic and allotted to the various treatment groups as described above. There were 10 animals per group. They were gavaged once daily as described above for 13 days. On Day 14, following an overnight fast, a drop of blood from the tail vein was collected for measuring baseline blood glucose level (Time 0). The animals were then given glucose (2 g glucose/kg BW) by gavage and blood was sampled from the tail vein at 10, 30 min, 60 min, and 120 min following the glucose challenge.

### Experiment 3 (Sucrose Challenge)

Animal model. Mice were made diabetic as described above and were allotted to eight groups (10 animals per group) as follows:

1) Control (saline, 0.2 ml/20 g BW)
2) Positive Control (acarbose, 2.5 mg/kg body weight)
3) PPF (50, 100, and 200 mg/kg body weight)

Treatment and Measurement. Test samples were prepared at appropriate concentrations so that the required doses were delivered in a volume of 0.2 mL/20 g body weight. Acarbose was also dissolved in 0.5% CMC (25 mg/ml) and given at a dose of 25 mg/kg BW. Ten minutes after gavaging of samples, blood was sampled from tail vein for glucose measurement (Time 0). The animals were then gavaged with sucrose (20% in water at a dose of 2 g/kg body weight). Blood was sampled from the tail vein at 10, 30 min, 60 min, and 120 min following the sucrose challenge for glucose measurement. A group of non-diabetic mice were given an equivalent volume of saline (0.2 ml/20 g BW).

### Results

During the study, non-diabetic mice gained about 19 g in body weight. This increase did not occur in STZ-diabetic mice. Treatment of STZ mice with test samples did not improve weight gain.

Serum Glucose. At the start of the treatment period (Day 0) serum glucose in STZ-diabetic mice was 15.02±3.0 mM compared to 5.71±0.9 mM in non-diabetic mice (p<0.05; see Table 9). The degree of hyperglycemia in diabetic mice increased by the end of the study to 22.1±2.8 mM. Metformin treatment significantly lower serum glucose at each of the time points (days 7, 14, 21, and 28) however, blood glucose remained elevated compared to non-diabetic levels. A. nodosum crude extract (CE) treatment resulted lowered serum glucose significantly on days 7, 14, and 21 (P<0.05), whereas the PSF produced lower serum glucose on days 7 and 14 (P<0.05) and PPF only on day 14.

Total cholesterol and glycated hemoglobin. STZ-diabetic mice had elevated total cholesterol and glycated Hb levels compared with the non-diabetic control group (Table 10). These were reduced by metformin, CE, and PPF.

Antioxidant activity. The blood total antioxidant level was decreased significantly in the STZ-diabetic mice compared to non-diabetic control mice (Table 10). Treatment with metformin or with the A. nodosum samples resulted in significant increases in antioxidant activity.

Liver glycogen. Glycogen level in liver was decreased in STZ-diabetic mice compared to the level in non-diabetic mice (4.41±0.92 versus 6.70±1.08 mg/g wet weight of liver tissue; Table 10). Metformin, and CE extract significantly increased liver glycogen level in STZ-diabetic mice.

Glucose tolerance test. Glucose tolerance is impaired in STZ-diabetic mice. Glucose tolerance testing, performed following 14 day of treatment with the A. nodosum fraction revealed that none of the fractions were effective in improving glucose tolerance (results not shown).

Sucrose challenge. Three different doses of PPF (50, 100, and 200 mg/kg BW) were tested in diabetic animals. For comparison, one group of mice was treated with Acarbose, an α-glucosidase inhibitor from Bayer. These test materials were given 10 minutes prior to the sucrose challenge. The results are shown in Table 11. Acarbose inhibited glucose absorption at each of the time points tested. An effect of PPF was evident at the 10- and 120-minute time points for each of the concentrations used. In STZ-mice, a sucrose challenge resulted in an area-under-the-curve (AUC) for glucose of 3494 units (mmol/L×120 min.) This was reduced in acarbose-treated mice by 33% (2340 units) and in PPF-treated mice by 12% (3076 units) and 17% (2899 units) for 200 and 50 mg/kg body weight doses, respectively.

TABLE 9
Effect of A. nodosum fractions on blood glucose in STZ-diabetic mice

| | | Dose | Blood glucose (mM) | | | | |
|---|---|---|---|---|---|---|---|
| Group | n | (mg/kg) | Day 0 | Day 7 | Day 14 | Day 21 | Day 28 |
| Non-diabetic | 10 | | 5.71 ± 0.9* | 5.91 ± 0.5* | 4.92 ± 0.7* | 5.59 ± 0.8* | 6.13 ± 1.0* |
| STZ-Control | 10 | 250 | 15.02 ± 3.0 | 16.61 ± 3.4 | 17.03 ± 2.7 | 16.24 ± 2.5 | 22.05 ± 2.8 |
| STZ-Metformin | 10 | 200 | 13.50 ± 2.5 | 13.04 ± 5.8* | 11.60 ± 4.1* | 12.38 ± 4.7* | 13.59 ± 7.7* |
| STZ-Crude extract | 10 | 200 | 15.09 ± 2.8 | 12.78 ± 3.7* | 12.43 ± 2.6* | 13.41 ± 3.2* | 22.12 ± 4.6 |
| STZ-polysaccharide | 10 | 200 | 13.04 ± 2.6 | 12.82 ± 4.4* | 13.38 ± 4.1* | 13.92 ± 7.1 | 19.53 ± 7.8 |
| STZ-polyphenolic | 10 | 200 | 14.42 ± 3.0 | 13.31 ± 5.5 | 11.66 ± 4.0* | 13.48 ± 6.6 | 16.92 ± 7.4 |

Results are the Mean ± S.D.
*Significant difference from the STZ-diabetic group at P < 0.05

TABLE 10
Effect of A. nodosum fractions on the biochemical parameters in STZ-Diabetic mice

| | | | | | | Liver |
|---|---|---|---|---|---|---|
| | | | Total | | AntIoxidant | glycogen |
| | | Dose | Cholesterol | Glycated Hb | activity | (mg/g wet |
| Group | n | (mg/kg) | (mmol/L) | mmol/L | (U/ml serum) | tissue) |
| Non-diabetic | 10 | — | 2.16 ± 0.4* | 4.94 ± 0.4* | 6.92 ± 1.2* | 6.70 ± 1.1* |
| STZ-Diabetic | 10 | 250 | 2.67 ± 0.4 | 6.20 ± 0.6 | 5.50 ± 1.2 | 4.41 ± 0.9 |
| STZ-Metformin | 10 | 200 | 1.93 ± 0.7* | 5.22 ± 0.5* | 8.15 ± 1.8* | 5.51 ± 0.8* |
| STZ-Crude extract | 10 | 200 | 1.99 ± 0.8* | 5.15 ± 0.4* | 8.54 ± 1.3* | 6.49 ± 1.6* |
| STZ-polysaccharide | 10 | 200 | 2.64 ± 0.5 | 6.23 ± 0.7 | 7.55 ± 1.6* | 5.57 ± 1.6 |
| STZ-polyphenolic | 10 | 200 | 2.00 ± 0.5* | 5.47 ± 0.6 | 7.56 ± 0.4* | 4.60 ± 1.2 |

Results are the Mean ± S.D.
*Significant difference from the STZ-diabetic group at P < 0.

TABLE 11
Effect of Ascophyllum nodosum PPF on sucrose tolerance in STZ-diabetic mice

| | | Dose | | | | | |
|---|---|---|---|---|---|---|---|
| Treatment Group | n | (mg/kg) | 0 min | 10 min | 30 min | 60 min | 120 min |
| Non-Diabetic | 10 | — | 5.53 ± 0.8* | 11.27 ± 2.0* | 9.84 ± 2.0* | 9.82 ± 2.5* | 7.46 ± 0.8* |
| STZ-Diabetic | 10 | | 17.68 ± 3.0 | 33.96 ± 5.8 | 29.78 ± 3.2 | 30.34 ± 6.1 | 26.21 ± 3.0 |
| Acarbose | 10 | 25 | 15.64 ± 2.6 | 21.71 ± 3.6* | 24.67 ± 4.1* | 19.80 ± 4.8* | 14.29 ± 2.9* |
| STZ-polyphenolic | 10 | 200 | 15.61 ± 3.0 | 27.46 ± 5.8* | 29.37 ± 4.8 | 26.31 ± 6.6 | 22.27 ± 4.7* |
| STZ-polyphenolic | 10 | 100 | 15.88 ± 2.7 | 26.20 ± 6.4* | 30.75 ± 6.1 | 30.37 ± 7.9 | 20.99 ± 6.3* |
| STZ-polyphenolic | 10 | 50 | 17.02 ± 2.3 | 24.43 ± 8.4* | 27.66 ± 7.6 | 25.71 ± 5.3* | 19.96 ± 6.8* |

Data are the mean ± standard deviation of serum glucose level in mmol/l.
*Significant different compared to value at the same time point for the Control STZ-Diabetic group (p < 0.05).

### EXAMPLE 4

This example demonstrates the structural elucidation of an extract enriched in polyphenolic compound by examining derivatization and degradation products.

### Ascophyllum Extraction Process

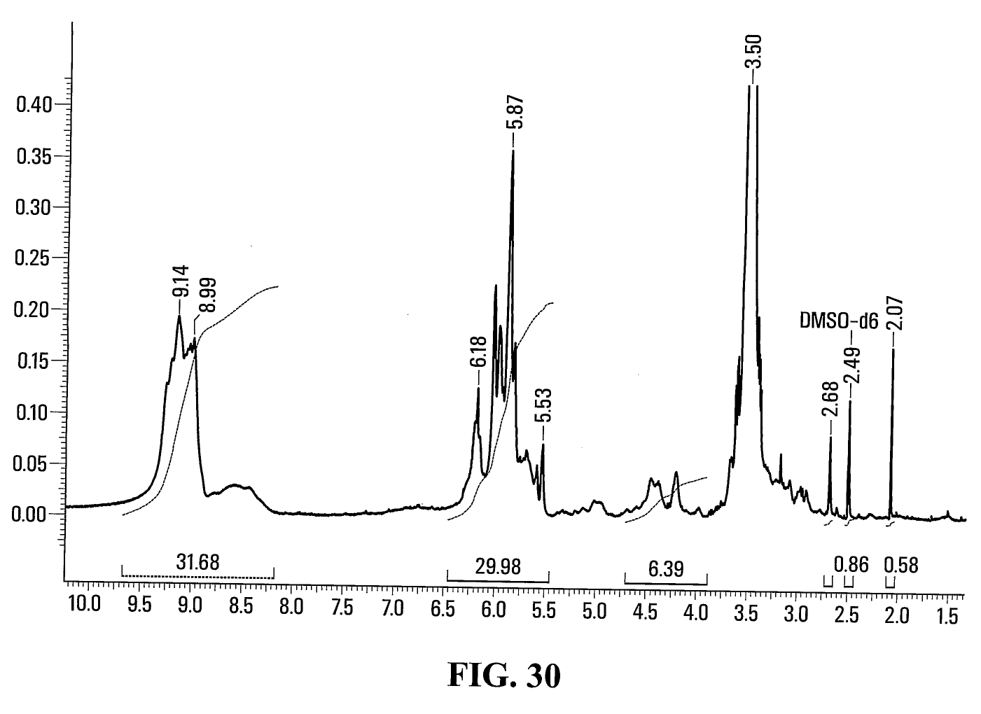

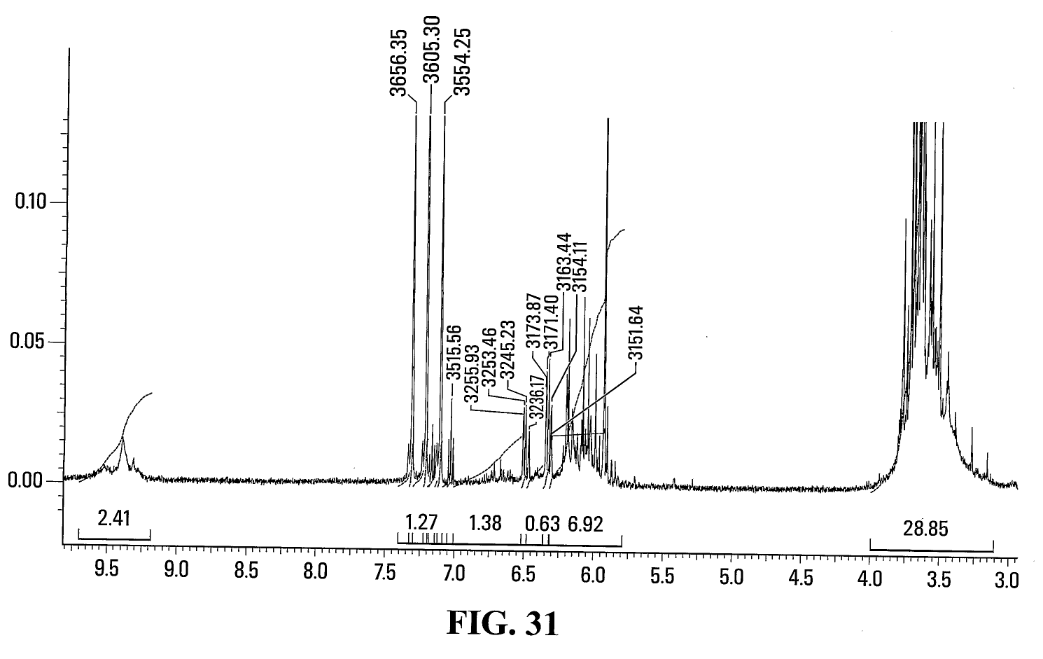

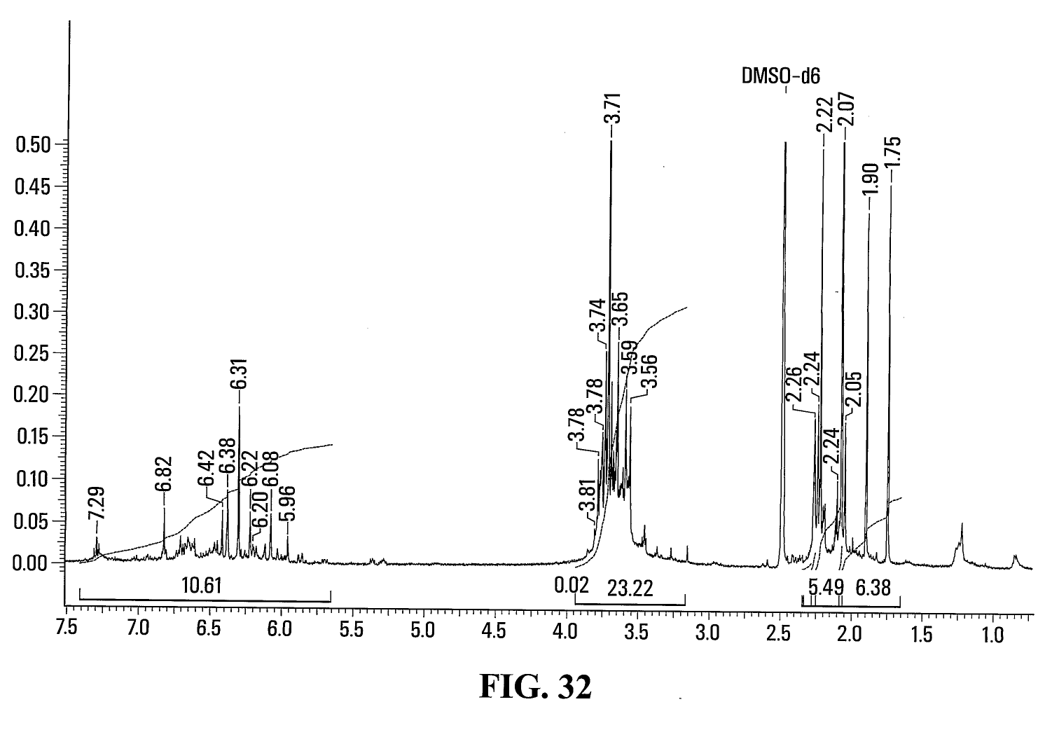

a) The Ascophyllum powder was extracted by 50% aqueous ethanol at room temperature and low molecular weight saccharides (such as mannitols) were obtained.
b) The extract was then separated into a low molecular weight fraction (LMW, <30K Dalton) and a high molecular weight fraction (HMW, >30K Dalton) by ultrafiltration. From ¹H NMR analysis, the LMW fraction is believed to be mannitols while the HMW fraction is believed to be phlorotannins with only bulk hydroxyls and aromatic protons shown in FIG. 30. The equal integrations for the regions of δ 8.0-9.5 ppm and δ 5.5-6.5 ppm shows approximately equal aryl and hydroxyl protons, which indicates mainly straight chain structures rather than branched ones.
c) Phase-transfer catalyzed methylation using dimethylsulphate in the presence of Adogen 464 was used to methylate the hydroxyl groups. The methylation was repeated three times to ensure no free hydroxyl groups remained.
d) The aryl ether bridges between phloroglucinol units were degradated by sodium metal in liquid ammonia at −70° C. This reaction should have no effect on the direct C—C linkages and protected hydroxyl groups as seen in the ¹H NMR spectrum of FIG. 31.
e) For further separation, a simple acetylation in acetic anhydride and pyridine was employed to acetylate the hydroxyl groups that resulted from the degradation of the aryl ether linkages. A ¹H NMR spectrum of the acetylated mixture is shown in FIG. 32.

### Structural Elucidation of Extract

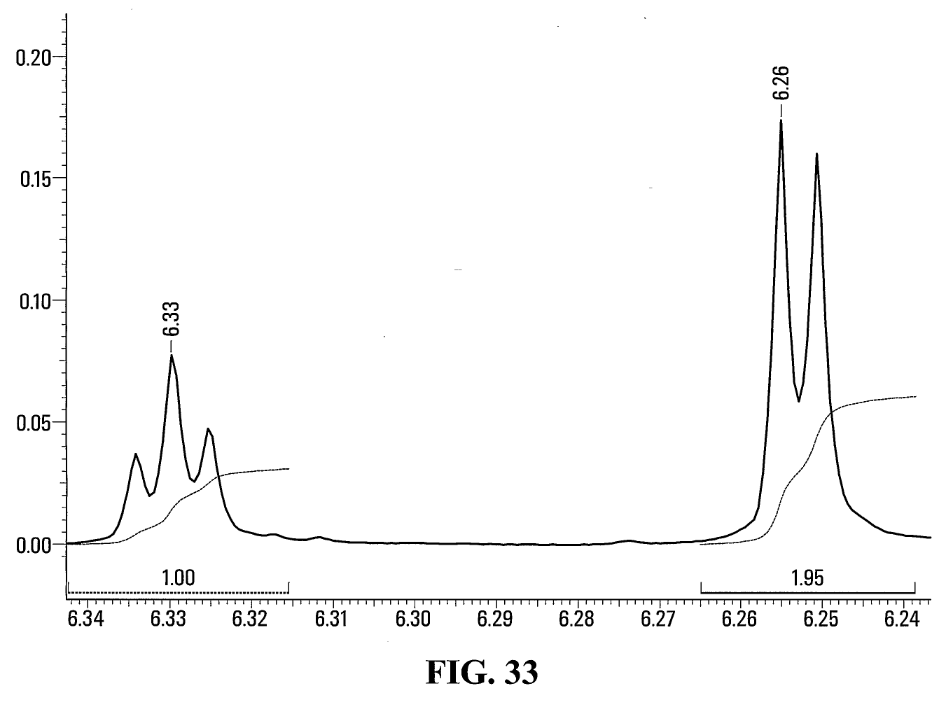

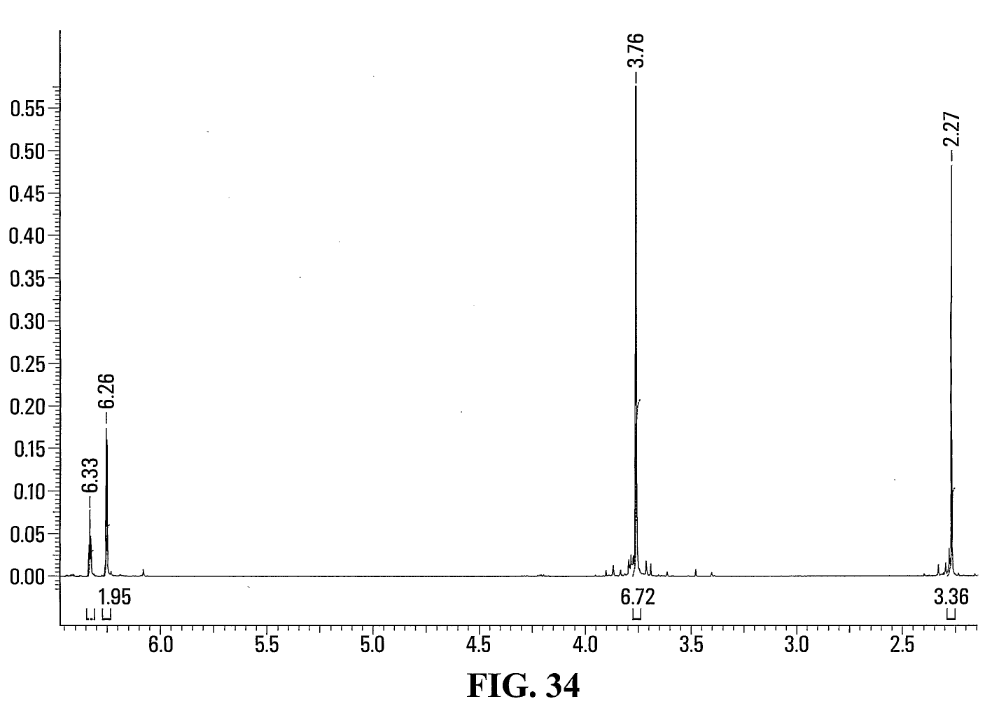

a) A silica gel column with gradient elution was used to separate the acetylated product. The elution began with a mobile phase of 100% chloroform which was incrementally changed to a 10% methanol/90% chloroform. The first fraction collected from the column was found to be a simple, pure compound from its ¹H NMR spectrum of FIG. 34. An expansion of the downfield region of the spectrum (FIG. 33) shows the triplet at δ 6.33 ppm and the doublet at δ 6.25 ppm are coupled to each other. The coupling constant was calculated as 2.2 Hz, typical of “W” coupling. Thus, the first fraction from the column is believed to be a single aromatic ring compound of the following structure:

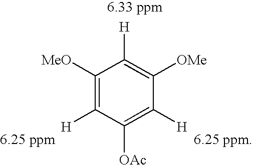

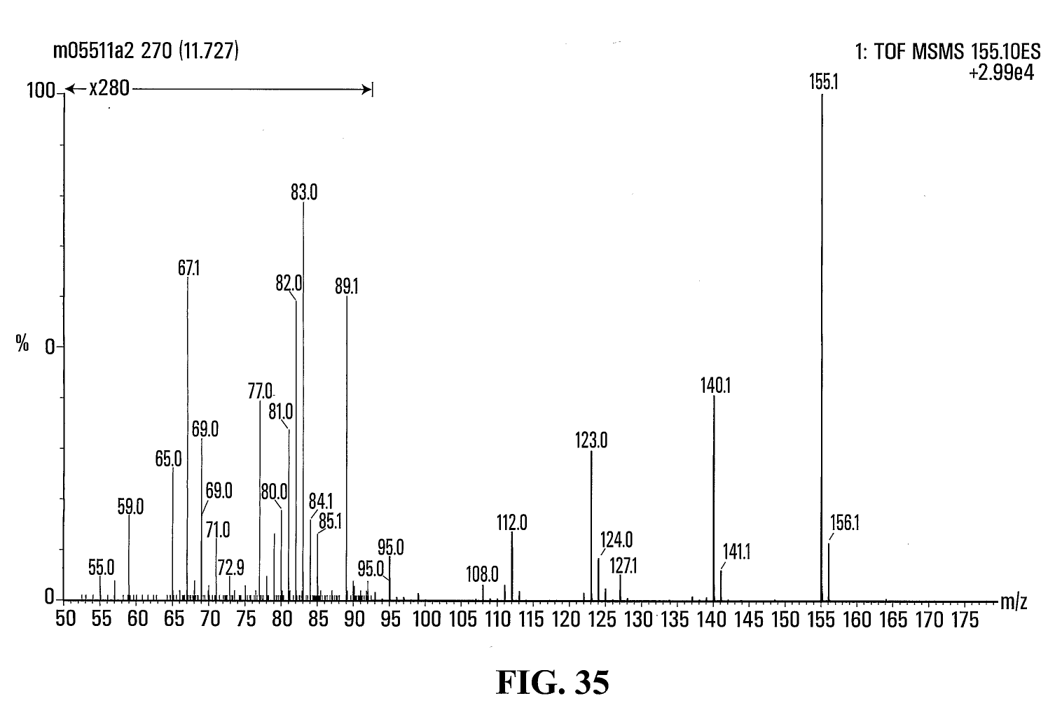

The structure of the above aromatic ring compound before acetylation is believed to be 3,5-dimethoxyphenol. The hydroxyl group arises from the broken C—O—C bridge after degradation by sodium metal. The structure is further identified by the LC-MS spectrum of FIG. 35 with an exact mass of 154.063.

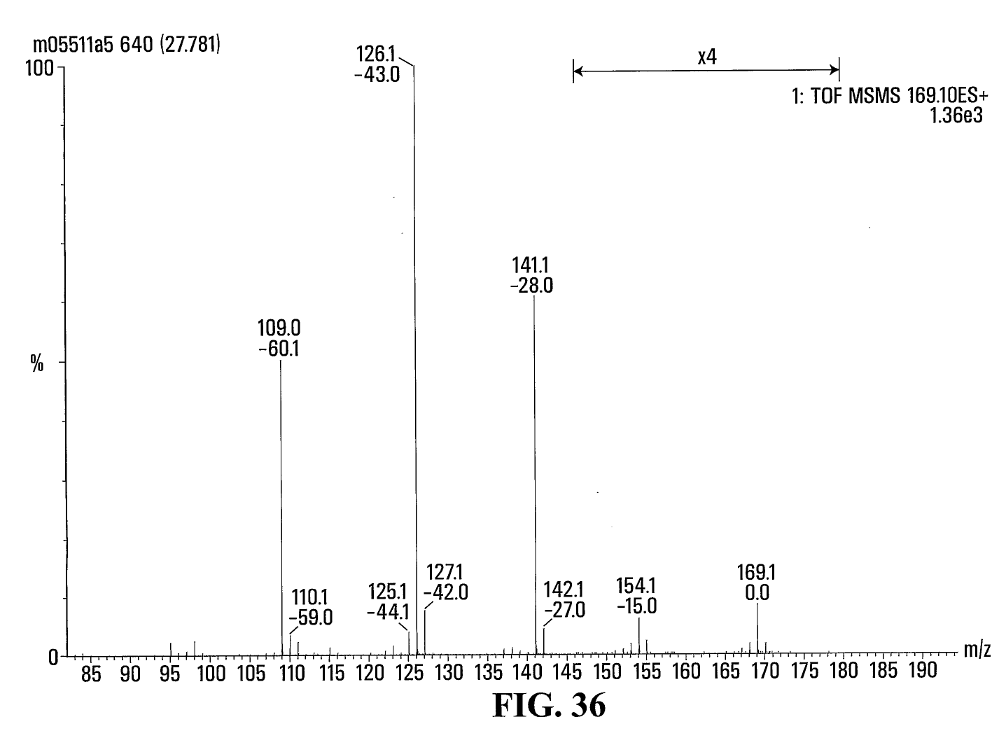

b) The LC-MS spectrum, m05511a5, shown in FIG. 36 has a fragment with an exact mass of 169.079. The exact mass suggests a molecular formula of C₉H₁₂O₃. Thus, the monomer is believed to be 1,3,5-trimethoxybenzene. This unit represents another linkage of phloroglucinol and fuhalols, which have not only meta-oxygen, but also ortho-oxygen as aryl ether bonds.

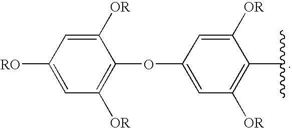

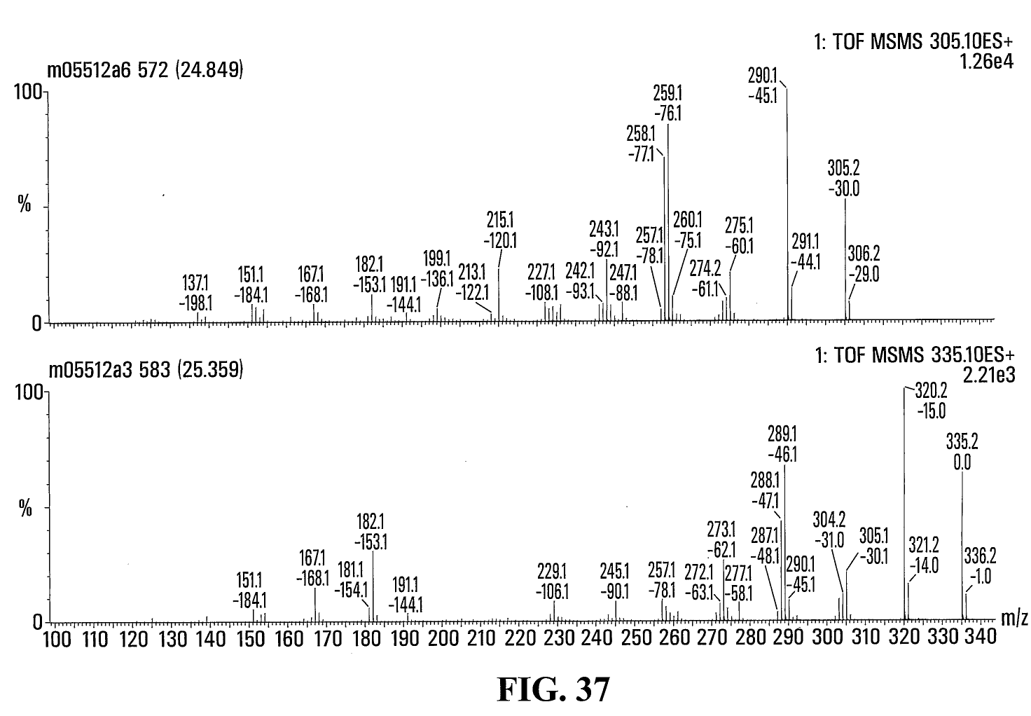

c) The positive and negative ion ESI spectra, m05512a6 and m05512a3, shown in FIG. 37 have two fragments that do not exhibit typical aryl ether bonding prevalent with many of the structures. They were found to have exact masses of 304.136 and 334.153, differing by a methoxy group. The evidence to support direct C—C linkages is the lack of response in the negative ion ESI spectrum, which favor O⁻ by losing the proton. Thus, the structure of the two fractions is believed to be the following:

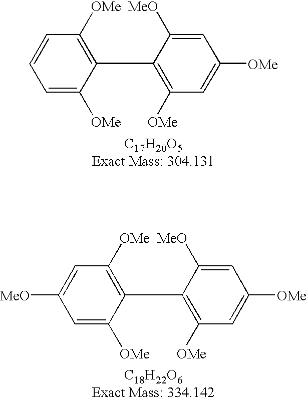

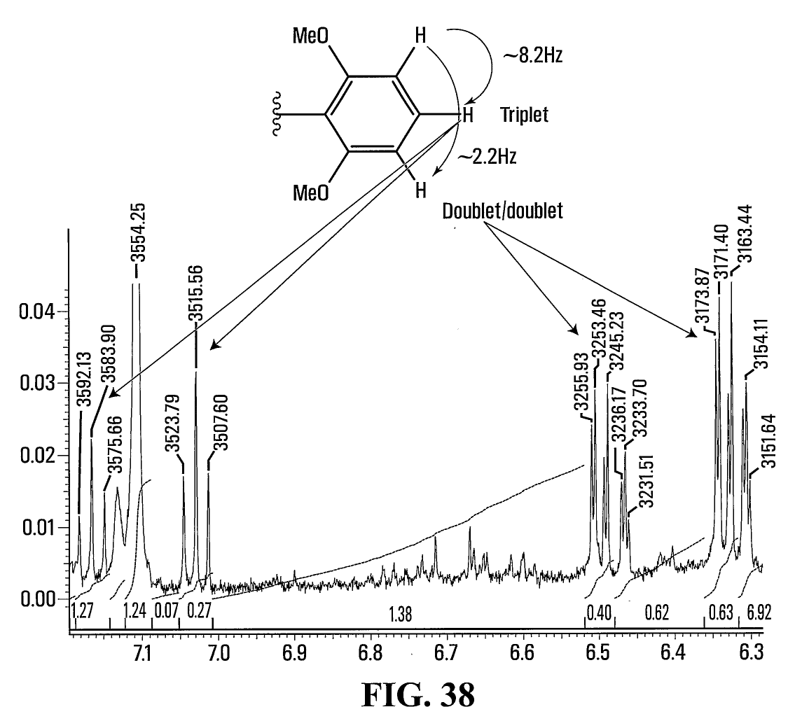

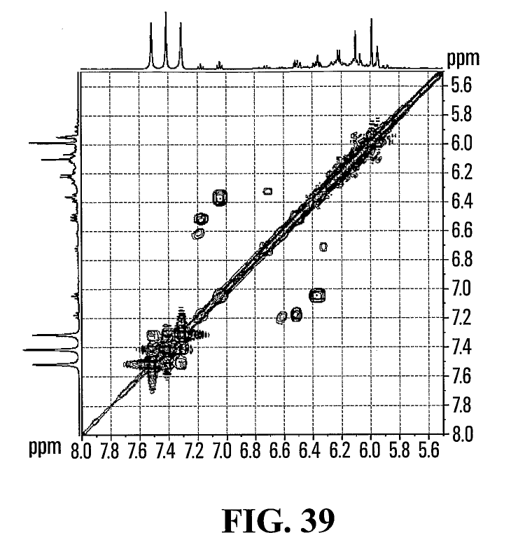

Further support for assigning the above chemical structure to the fraction identified as C₁₇H₂₀O₅ comes from the ¹H NMR spectrum (FIG. 38) and the COSY NMR spectra (FIG. 39). The correlation peaks of the COSY spectrum supports resonance between the three neighbouring protons.

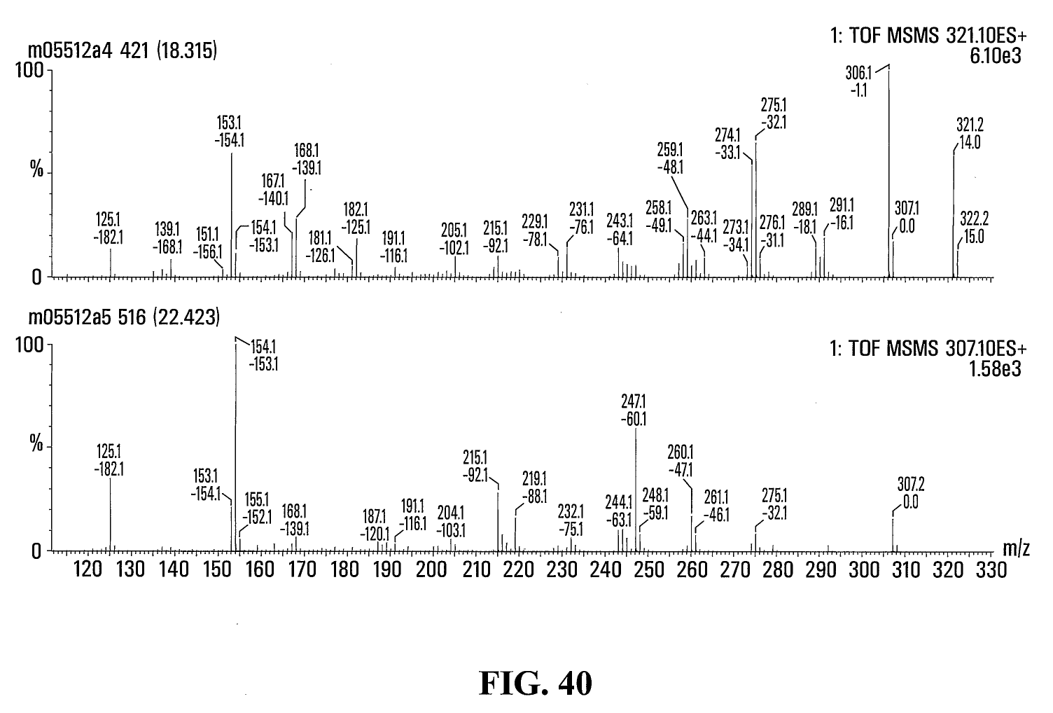

d) The positive and negative ion ESI spectra, m05512a4 and m05512a5, shown in FIG. 40 have two fragments of interest with exact masses of 306.110 and 320.126. Two possible isobaric structures are proposed for each compound. One is the aryl ether structure (below left) and the other one is C—C direct bond between the benzenoid rings (below right).

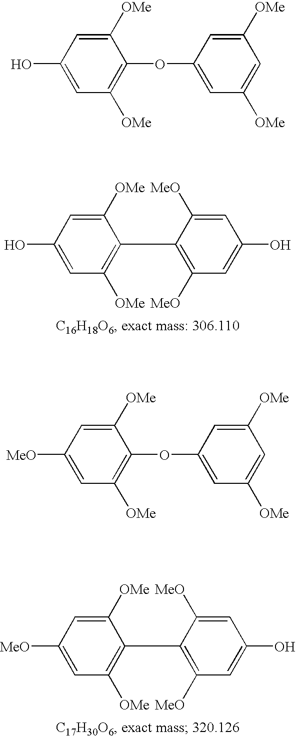

A closer look at the difference between the two ESI spectra at mass 320 indicates the presence of a free hydroxyl group on the non-aryl ether bonded structure. That explains the large difference in response between positive and negative ion acquisitions.

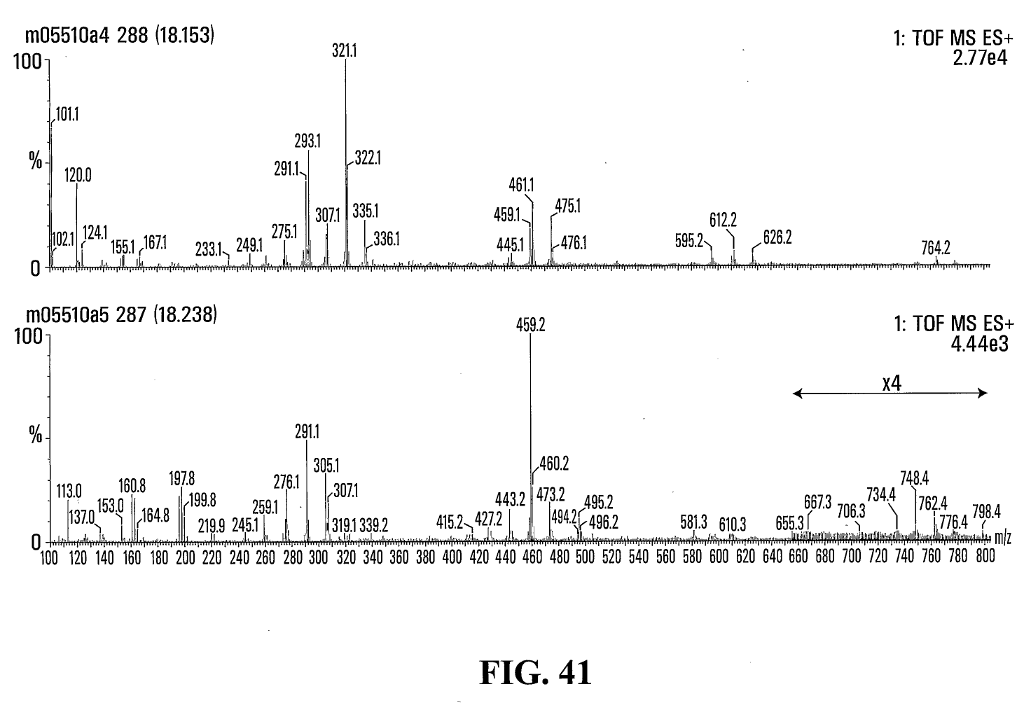

e) The positive and negative ion ESI spectra, m05510a4 and m05510a5, shown in FIG. 41 has a significant fragment at 459.2 m/z (positive ESI), which has an even stronger negative ion counterpart. Again this supports the presence of free hydroxyl groups in the compounds. Thus, three possible structures of the fragment are proposed herein. Each one has three aromatic rings and may contain aryl ether bond or direct C—C linkage or both.

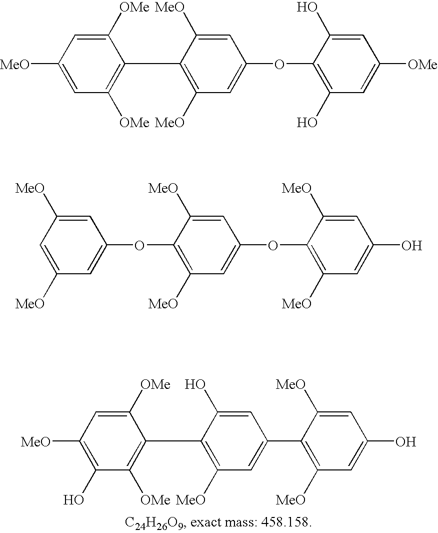

### REFERENCES

Fukumoto, L. R. & G. Mazza. 2000. Assessing antioxidant and prooxidant activities of phenolic compounds. Journal of Agricultural and Food Chemistry 48: 3597-3604.
Glombitza, K. W. & K. Klapperich. 1985. Antibiotics from Algae. XXXIV. Cleavage of the high-molecular-weight methylated phlorotannin fraction from the brown alga Pelvetia canaliculata. Botanica Marina, 18:139-144.
McLnnes, A. G., M. A. Ragan, D. G. Smith & J. A. Walter. 1984. High-molecular-weight phloroglucinol-based tannins from brown algae: structural variants. Proceeding of International Seaweed Symposium, 11:597-602.
Rininger J A, S. Kickner, P. Chigurupati, A. McLean & Z. Franck. 2000. Immunopharmacological activity of Echinacea preparations following simulated digestion on murine macrophages and human peripheral blood mononuclear cells. J Leukoc Biol 68:503-10
Sawada, Y., T. Tsuno, T. Ueki, H. Yamamoto, Y. Fukagawa & T. Oki. 1993. Pradimicin Q, a new pradimicin aglycone, with α-glucosidase inhibitory activity. Journal of Antibiotics, 46, 507-10.
Singleton, V. L., R. Orthofer & R. M. Lamuela-Raventos, 1999. Analysis of total phenols and other oxidation substrates and antioxidants by means of Folin-Ciocalteu reagent. In Abelson, J. N., M. Simon & H. Sies (eds), Methods in Enzymology, Volume 299: Oxidants and antioxidants, Part A. Academic Press, Orlando: 152-178.
Zhang, Q., J. Zhang, J. Shen, A. Silva, D. Dennis & C. Barrow. 2004. A simple 96-well microplate method for estimation of total polyphenol content in seaweeds. XVIII International Seaweed Symposium, Bergen, Norway, June 20-25. Poster No. 260.
Zhang, Q., J. Zhang, A. Silva, J. Shen, D. Dennis & C. J. Barrow. 2004. Determination of sulfated polysaccharide content in marine algae using calorimetric method. Marine Biopolymers: structure, functionality and applications. A satellite to the XVIII International Seaweed Symposium. June 27-29, Trondheim, Norway. Poster No. 11.

All publications and patent applications cited in this specification are herein incorporated by reference as if each individual publication or patent application were specifically and individually indicated to be incorporated by reference. The citation of any publication is for its disclosure prior to the filing date and should not be construed as an admission that the present invention is not entitled to antedate such publication by virtue of prior invention.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

As used in this specification and the appended claims, the singular forms “a,” “an,” and “the” include plural reference unless the context clearly dictates otherwise. Unless defined otherwise all technical and scientific terms used herein have the same meaning as commonly understood to one of ordinary skill in the art to which this invention belongs.

1. An extract of Ascophyllum, comprising at least about 20% by weight of polyphenolic compounds having a molecular weight of about 30 kDa to about 830 kDa.

2-9. (canceled)

10. The extract according to claim 1, wherein said molecular weight is about 40 kDa to about 300 kDa.

11. The extract according to claim 1, wherein said polyphenolic compounds have an average molecular weight of about 100 kDa.

12. The extract according to claim 1, wherein said polyphenolic compounds comprise phenol groups linked by ether bonds and/or phenol groups linked by carbon-carbon bonds.

13. The extract according to claim 1, wherein said polyphenolic compounds comprise highly polymerized phloroglucinol-based tannins (phlorotannins).

14. The extract according to claim 13, wherein said phlorotannins have a degree of polymerization (DP) of about 200 to about 8000.

15. The extract according to claim 13, wherein said phlorotannins have a degree of polymerization (DP) of about 200 to about 3000.

16. The extract according to claim 13, wherein said phlorotannins comprise either or both monomers of formulas:

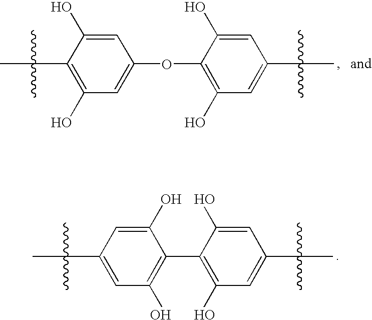

17. The extract according to claim 1, wherein said Ascophyllum is selected from Ascophyllum nodosum, Ascophyllum laevigatum and Ascophyllum mackayi.

18. The extract according to claim 1, wherein said Ascophyllum is Ascophyllum nodosum.

19. An extract of Ascophyllum, comprising at least about 40% by weight of sulfated polysaccharides having a molecular weight of about 100 kDa to about 3000 kDa, and obtained by an extraction process comprising extracting Ascophyllum with water at a temperature of about 80° C.

20-23. (canceled)

24. The extract according to claim 19, wherein said sulfated polysaccharides have an average molecular weight of about 300 kDa to about 1000 kDa.

25. The extract according to claim 19, wherein said sulfated polysaccharides have a sulfation level of about 5% to about 30%.

26. The extract according to claim 19, wherein said sulfated polysaccharides comprise a monosaccharide selected from the group consisting of glucose, fructose, mannose, galactose, ribose, arabinose, xylose, lyxose, erythrose and threose.

27. The extract according to claim 19, wherein said Ascophyllum is selected from Ascophyllum nodosum, Ascophyllum laevigatum and Ascophyllum mackayi.

28. The extract according to claim 19, wherein said Ascophyllum is Ascophyllum nodosum.

29. The extract according to claim 1, further comprising sulfated polysaccharides having a molecular weight of about 100 kDa to 3000 kDa.

30. The extract according to claim 1, obtained by an extraction process comprising extracting Ascophyllum with an aqueous organic solvent to form an aqueous organic extract.

31-54. (canceled)

55. A composition comprising an extract according to claim 1 and a carrier, which composition is a nutritional supplemental or a food product.

56-58. (canceled)

59. The composition according to claim 55, wherein the composition is the nutritional supplement in the form of a sachet.

60-86. (canceled)
